(19) United States
(12) Patent Application Publication
(10) Pub. No.: US 2026/0125412 A1
(43) Pub. Date: May 7, 2026

(54) NUCLEOTIDES WITH ALPHA-IMINO PHOSPHATE GROUPS AND BRIDGING SULFURS, AND METHODS OF SYNTHESIZING AND USING THE SAME

(71) Applicant: Illumina, Inc., San Diego, CA (US)

(72) Inventors: Jean-Alexandre Richard, Singapore (SG); Ramesh Neelakandan, Singapore (SG); Sadeer Salam, Singapore (SG); Min Yen Lee, Singapore (SG); Daniel Hartoyo Lukamto, Singapore (SG); Shi Yuan Jeow, Singapore (SG); Xi Liew, Singapore (SG); Jin Yong Neo, Singapore (SG); Tsz Ying Yuen, Singapore (SG); Xiangyuan Yang, Singapore (SG); Sharyuen Soh, Singapore (SG); Jing Liang, Singapore (SG); Deepti Sharma, Singapore (SG); Yin Nah Teo, Singapore (SG)

(73) Assignee: Illumina, Inc., San Diego, CA (US)

(21) Appl. No.: 19/379,047

(22) Filed: Nov. 4, 2025

Related U.S. Application Data
(60) Provisional application No. 63/716,875, filed on Nov. 6, 2024.

(51) Int. Cl. C07H 19/073; C07H 19/173; C12P 19/34

(52) U.S. Cl. CPC C07H 19/073; C07H 19/173; C12P 19/34

(57) ABSTRACT

Nucleotides with alpha-imino phosphate groups and bridging sulfurs, and methods of synthesizing and using the same, are provided herein. In some examples, a nucleotide may include a sugar, a nucleobase coupled to the sugar, an alpha-imino phosphate group, and a bridging sulfur coupling the alpha-imino phosphate group to the sugar.

### CROSS-REFERENCE TO RELATED APPLICATIONS

This application claims the benefit of U.S. Provisional Patent Application No. 63/716,875, filed Nov. 6, 2024 and entitled “NUCLEOTIDES WITH ALPHA-IMINO PHOSPHATE GROUPS AND BRIDGING SULFURS, AND METHODS OF SYNTHESIZING AND USING THE SAME,” the entire contents of which are incorporated by reference herein.

### FIELD

This application relates to nucleotides.

### BACKGROUND

Modified nucleoside triphosphates are key drivers of various sequencing technologies. For example, some sequencing technologies track the incorporation of fluorophore-labelled nucleotides into a polynucleotide. The sequence of the polynucleotide is determined by reading the emission from nucleotide-specific fluorophores during each incorporation cycle.

It may be desirable to modify nucleotides to include different moieties. Traditional methods of installing modifications may use intermediates that are costly and involve multi-step syntheses. Moreover, extensive functionalization of triphosphate substrates carries a risk of phosphate chain degradation. Such degradation can lead to poor overall yields.

### SUMMARY

Nucleotides with alpha-imino phosphate groups and bridging sulfurs, and methods of synthesizing and using the same, are provided herein.

Some examples herein provide a nucleotide. The nucleotide may include a sugar; a nucleobase coupled to the sugar; an alpha-imino phosphate group; and a bridging sulfur coupling the alpha-imino phosphate group to the sugar.

In some examples, the nucleotide may include a sulfonyl group or aryl group coupled to the alpha-imino phosphate group. In some examples, the nucleotide further may include a first functional group coupled to the alpha-imino phosphate group via the sulfonyl group or aryl group. In some examples, the aryl group is functionalized. In some examples, the nucleotide includes a second functional group coupled to the nucleobase. The second functional group may be of a same type as the first functional group. Or, the second functional group may be of a different type than the first functional group. In some examples, the second functional group further is coupled to the first functional group to form a loop. In some examples, the nucleobase includes a purine analog or a pyrimidine analog.

In some examples, the nucleobase includes a purine, a pyrimidine, a purine analog, or a pyrimidine analog.

In some examples, the sugar includes ribose or deoxyribose.

In some examples, the nucleotide includes at least one phosphate group coupled to the alpha-imino phosphate group.

In some examples, the alpha-imino phosphate group is part of a triphosphate group.

Some examples herein provide a kit. The kit may include any of the nucleotides provided herein, and a reagent for selectively cleaving the bond between the alpha-imino phosphate and the bridging sulfur.

In some examples, the reagent includes an Ag(I) metal salt, Au(III) metal salt, Pd(II) metal salt, or Oxone.

In some examples, the kit further includes a polymerase for incorporating the nucleotide into a polynucleotide.

Some examples herein provide a method for modifying a polynucleotide. The method may include using a polymerase to incorporate any of the nucleotides provided herein into a polynucleotide. The method may include using a reagent to selectively cleave the bond between the alpha-imino phosphate and the bridging sulfur after the nucleotide is incorporated into the polynucleotide.

Some examples herein provide a method for modifying a polynucleotide. The method may include installing a thiol group at a 5′ position of a sugar of the nucleotide; coupling a phosphor atom to the sulfur atom of the thiol group; and forming an alpha-imino phosphate group coupled to a sulfonyl group or an aryl group.

In some examples, installing the thiol group includes using a Mitsonobu reaction followed by reduction.

In some examples, forming the alpha-imino phosphate group includes using a phosphoramidite.

In some examples, forming the alpha-imino phosphate group includes using a Staudinger reaction.

In some examples, the sulfonyl group or the aryl group is coupled to a first functional group. In some examples, the nucleotide includes a nucleobase including a second functional group. In some examples, the method further includes coupling the first functional group to the second functional group to form a loop.

Some examples herein provide a method for modifying a polynucleotide. The method may include, in examples in which the sulfonyl group or the aryl group is coupled to a first functional group, coupling the first functional group to the second functional group to form a loop.

It is to be understood that any respective features/examples of each of the aspects of the disclosure as described herein can be implemented together in any appropriate combination, and that any features/examples from any one or more of these aspects can be implemented together with any of the features of the other aspect(s) as described herein in any appropriate combination to achieve the benefits as described herein.

### BRIEF DESCRIPTION OF DRAWINGS

FIG. 1 schematically illustrates nonlimiting examples of the present nucleotides with alpha-imino phosphate groups and bridging sulfurs.

FIG. 2 schematically illustrates an example manner in which the alpha-imino phosphate group and bridging sulfur stabilize the present nucleotides.

FIGS. 3A-3C schematically illustrate example modified nucleobases, and functional groups, that may be used in the present nucleotides.

FIG. 4A schematically illustrates an example method of synthesizing deoxythymidine with a sulfonyl group coupled to the alpha-imino phosphate group and a 5′-S-phosphorothiolate bridging sulfur.

FIG. 4B schematically illustrates another example method of synthesizing deoxycytidine with a sulfonyl group coupled to the alpha-imino phosphate group and a 5′-S-phosphorothiolate bridging sulfur.

FIG. 4C schematically illustrates another example method of synthesizing deoxyadenosine with a sulfonyl group coupled to the alpha-imino phosphate group and a 5′-S-phosphorothiolate bridging sulfur.

FIG. 4D schematically illustrates another example method of synthesizing deoxyguanosine with a sulfonyl group coupled to the alpha-imino phosphate group and a 5′-S-phosphorothiolate bridging sulfur.

FIG. 4E illustrates another example method of synthesizing a nucleotide with a sulfonyl group coupled to the alpha-imino phosphate group and a 5′-S-phosphorothiolate bridging sulfur.

FIG. 5 schematically illustrates an example method of synthesizing deoxythymidine with a phenyl group coupled to the alpha-imino phosphate group and a 5′-S-phosphorothiolate bridging sulfur.

FIG. 6 illustrates a flow of operations in an example method of synthesizing a nucleotide with an alpha-imino phosphate group and a bridging sulfur.

FIG. 7 illustrates a flow of operations in an example method of coupling a pyrophosphate group to an alpha-imino phosphate group.

FIG. 8 illustrates a flow chart of operations in an example method of using a nucleotide with an alpha-imino phosphate group and a bridging sulfur.

FIG. 9 schematically illustrates operations in an example method for using a nucleotide with an alpha-imino phosphate group and a bridging sulfur.

FIGS. 10A-10C respectively illustrate mass spectrometry (MS), high-performance liquid chromatography (HPLC), and ³¹P nuclear magnetic resonance (NMR) data for deoxythymidine synthesized in accordance with FIG. 4A.

FIGS. 11A-11B respectively illustrate MS and HPLC data for deoxycytidine synthesized in accordance with FIG. 4B.

FIGS. 12A-12B respectively illustrate MS and HPLC data for deoxyadenosine synthesized in accordance with FIG. 4C.

FIGS. 13A-13B respectively illustrate MS and HPLC data for deoxyguanosine synthesized in accordance with FIG. 4D.

FIGS. 14A-14B respectively illustrate MS and HPLC data for deoxythymidine synthesized in accordance with FIG. 4E.

FIG. 15A schematically illustrates hydrolysis of an example nucleotide triphosphate lacking an alpha-imino phosphate group and bridging sulfur.

FIG. 15B schematically illustrates the percent triphosphate as a function of time at room temperature, for the example nucleotide triphosphate of FIG. 15A.

FIG. 16A illustrates the percent triphosphate as a function of time, at room temperature, for example nucleotide triphosphates including an alpha-imino phosphate group and bridging sulfur.

FIG. 16B illustrates the percent triphosphate as a function of time, at elevated temperature, for example nucleotide triphosphates including an alpha-imino phosphate group and bridging sulfur.

FIGS. 17A-17D are images of electrophoretic gels showing polymerase incorporation of example nucleotides including alpha-imino phosphate groups and bridging sulfurs.

FIG. 18 is an image of an electrophoretic gel showing reagent-caused cleavage of an incorporated example nucleotide including an alpha-imino phosphate group and bridging sulfur.

### DETAILED DESCRIPTION

Disclosed herein are nucleotides with alpha-imino phosphate groups and bridging sulfurs, and methods of synthesizing and using the same. It may be desirable to incorporate modifications into nucleotides. For example, modifying the nucleotides may allow a sequence of the nucleotides to be identified optically or electronically.

As noted above, some previous methods for synthesizing modified nucleotides use advanced intermediates that are costly and involve multi-step syntheses. Other previous methods involve the functionalization of triphosphate substrates. This can lead to phosphate chain degradation, which in turn may lead to poor overall yields. In comparison, the presently disclosed nucleotides may be made in commercially usable yields, and may have improved stability over other types of nucleotides.

First, example terminology will be introduced. Then, nonlimiting examples of the present nucleotides including alpha-imino phosphates and bridging sulfurs, and methods of making the same, will be described. Then, data illustrating the synthesis and example uses of the present nucleotides will be provided.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but can include additional steps. When used in the context of a compound, composition, or device, the term “comprising” means that the compound, composition, or device includes at least the recited features or components, but can also include additional features or components.

The terms “substantially”, “approximately”, and “about” used throughout this Specification are used to describe and account for small fluctuations, such as due to variations in processing. For example, they can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

As used herein, terms such as “covalently coupled” or “covalently bonded” refer to the forming of a chemical bond that is characterized by the sharing of pairs of electrons between atoms. For example, a covalently coupled molecule refers to a molecule that forms chemical bonds with a substrate, as compared to coupling to the surface via other means, for example, a non-covalent bond such as electrostatic interaction.

The term “halogen” or “halo,” as used herein, means fluorine, chlorine, bromine, or iodine, with fluorine and chlorine being examples.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” or “C₁₋₄alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

Groups that include an alkenyl group include optionally substituted alkenyl, cycloalkenyl, and heterocycloalkenyl groups.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” or “C₂₋₄alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

Groups that include an alkynyl group include optionally substituted alkynyl, cycloalkynyl, and heterocycloalkynyl groups.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some examples, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl. Aryl groups (such as phenyl) optionally may be substituted, e.g., using one or more halogens (such as fluorine), alkoxy, or amine.

As used herein, “heterocycle” refers to a cyclic compound which includes atoms of carbon along with another atom (heteroatom), for example nitrogen, oxygen or sulfur. Heterocycles may be aromatic (heteroaryl) or aliphatic. An aliphatic heterocycle may be completely saturated or may contain one or more or two or more double bonds, for example the heterocycle may be a heterocycloalkyl. The heterocycle may include a single heterocyclic ring or multiple heterocyclic rings that are fused.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some examples, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” or “cycloalkene” means a carbocyclyl ring or ring system having at least one double bond, wherein no ring in the ring system is aromatic. An example is cyclohexenyl or cyclohexene. Another example is norbornene or norbornenyl.

As used herein, “heterocycloalkenyl” or “heterocycloalkene” means a carbocyclyl ring or ring system with at least one heteroatom in ring backbone, having at least one double bond, wherein no ring in the ring system is aromatic. In some examples, heterocycloalkenyl or heterocycloalkene ring or ring system is 3-membered, 4-membered, 5-membered, 6-membered, 7-membered, 8-membered, 9-membered, or 10-membered.

As used herein, “cycloalkynyl” or “cycloalkyne” means a carbocyclyl ring or ring system having at least one triple bond, wherein no ring in the ring system is aromatic. An example is cyclooctyne. Another example is bicyclononyne. Another example is dibenzocyclooctyne (DBCO).

As used herein, “heterocycloalkynyl” or “heterocycloalkyne” means a carbocyclyl ring or ring system with at least one heteroatom in ring backbone, having at least one triple bond, wherein no ring in the ring system is aromatic. In some examples, heterocycloalkynyl or heterocycloalkyne ring or ring system is 3-membered, 4-membered, 5-membered, 6-membered, 7-membered, 8-membered, 9-membered, or 10-membered.

As used herein, “heterocycloalkyl” means a non-aromatic cyclic ring or ring system containing at least one heteroatom in the ring backbone. Heterocycloalkyls may be joined together in a fused, bridged or spiro-connected fashion. Heterocycloalkyls may have any degree of saturation provided that at least one heterocyclic ring in the ring system is not aromatic. The heterocycloalkyl group may have 3 to 20 ring members (i.e., the number of atoms making up the ring backbone, including carbon atoms and heteroatoms), although the present definition also covers the occurrence of the term “heterocycloalkyl” where no numerical range is designated. The heterocycloalkyl group may also be a medium size heterocycloalkyl having 3 to 10 ring members. The heterocycloalkyl group could also be a heterocycloalkyl having 3 to 6 ring members. The heterocycloalkyl group may be designated as “3-6 membered heterocycloalkyl” or similar designations. In some six membered monocyclic heterocycloalkyls, the heteroatom(s) are selected from one up to three of O, N or S, and in some five membered monocyclic heterocycloalkyls, the heteroatom(s) are selected from one or two heteroatoms selected from O, N, or S. Examples of heterocycloalkyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, the term “nucleoside” is intended to mean a molecule that includes a sugar and at least one phosphate group, and in some examples also includes a nucleobase. A nucleoside that lacks a nucleobase can be referred to as “abasic.” Nucleosides include deoxyribonucleosides, modified deoxyribonucleosides, ribonucleosides, modified ribonucleosides, peptide nucleosides, modified peptide nucleosides, modified phosphate sugar backbone nucleosides, and mixtures thereof. Examples of nucleosides include adenosine, thymidine, cytidine, guanosine, uridine, deoxyadenosine, deoxythymidine, deoxycytidine, deoxyguanosine, and deoxyuridine.

As used herein, the term “nucleoside” also is intended to encompass any nucleoside analogue which is a type of nucleoside that includes a modified nucleobase and/or sugar compared to naturally occurring nucleosides. Example modified nucleobases include inosine, xanthine, hypoxanthine, 5-methylcytosine, 5-hydroxymethyl cytosine, 2-aminoadenine, 6-methyl adenine, 6-methyl guanine, 4-thiouracil, 8-hydroxyl adenine or guanine, 7-methylguanine, 7-methyladenine, 8-azaguanine, 8-azaadenine, or the like. Example modified nucleobases also include isocytosine, isoguanine, 2-aminopurine 2-propyl guanine, 2-propyl adenine, 2-thiouracil, 2-thiothymine, 2-thiocytosine, 5-thiouracil, 5-halocytosine, 5-propynyl uracil, 5-propynyl cytosine, 6-azo uracil, 6-azo cytosine, 6-azo thymine, 5-thiouracil, 8-halo adenine or guanine, 8-amino adenine or guanine, 8-thiol adenine or guanine, 8-thioalkyl adenine or guanine 5-halo substituted uracil or cytosine, 7-deazaguanine, 7-deazaadenine, 3-deazaguanine, 3-deazaadenine or the like.

As used herein, the term “nucleotide” is intended to mean a molecule that includes a sugar and at least one of: a phosphate group, a phosphoramidate, and a phosphorothioate. In some examples a nucleotide also includes a nucleobase. A nucleotide that lacks a nucleobase can be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides adenosine triphosphate (ATP), thymidine triphosphate (TTP), cytidine triphosphate (CTP), guanosine triphosphate (GTP), uridine triphosphate (UTP), deoxyadenosine triphosphate (dATP), deoxythymidine triphosphate (dTTP), deoxycytidine triphosphate (dCTP), deoxyguanosine triphosphate (dGTP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, 5-methylcytosine, 5-hydroxymethyl cytosine, 2-aminoadenine, 6-methyl adenine, 6-methyl guanine, 4-thiouracil, 8-hydroxyl adenine or guanine, 7-methylguanine, 7-methyladenine, 8-azaguanine, 8-azaadenine, or the like. Example modified nucleobases also include isocytosine, isoguanine, 2-aminopurine 2-propyl guanine, 2-propyl adenine, 2-thiouracil, 2-thiothymine, 2-thiocytosine, 5-thiouracil, 5-halocytosine, 5-propynyl uracil, 5-propynyl cytosine, 6-azo uracil, 6-azo cytosine, 6-azo thymine, 5-thiouracil, 8-halo adenine or guanine, 8-amino adenine or guanine, 8-thiol adenine or guanine, 8-thioalkyl adenine or guanine 5-halo substituted uracil or cytosine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides can include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), locked nucleic acid (LNA), and analogues thereof. A polynucleotide can be a single stranded sequence of nucleotides, such as RNA or single stranded DNA, a double stranded sequence of nucleotides, such as double stranded DNA, DNA that is folded to form a hairpin that is partially single stranded and partially double stranded, double-stranded amalgamations in which there are molecules that are non-covalently coupled to one another (e.g., via reversible hydrogen binding), and/or can include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides can include non-naturally occurring DNA, such as enantiomeric DNA. The precise sequence of nucleotides in a polynucleotide can be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I(Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

The terms “polynucleotide” and “oligonucleotide” are used interchangeably herein. The different terms are not intended to denote any particular difference in size, sequence, or other property unless specifically indicated otherwise. For clarity of description the terms can be used to distinguish one species of polynucleotide from another when describing a particular method or composition that includes several polynucleotide species.

As used herein, the term “polymer” refers to a molecule including many repeated subunits or recurring units. Non-limiting examples of polymer structures include linear, branched, or hyper-branched polymers. Polymers as described herein can be linear, branched, hyper-branched or dendritic. Different classes of polymer backbones include, but are not limited to, polyacrylamides, polyacrylates, polyurethanes, polysiloxanes, silicones, polyacroleins, polyphosphazenes, polyisocyanates, poly-ols, polysaccharides, polypeptides, and combinations thereof. A polymer can include one or more moieties that can react with one or more other moieties to form a covalent bond.

As used herein, the term “imino phosphate group” is intended to refer to a phosphate group which contains the moiety P═N, where the N may be bonded to another group and the P may be bonded to one or more other groups, such as to one or more oxygens and/or one or more other phosphate groups. An imino phosphate group alternatively may be referred to as a phosphazene or iminophosphorane.

As used herein, the term “bridging sulfur” is intended to refer to a sulfur atom that couples the sugar of a nucleotide to the alpha-phosphate group of the nucleotide (e.g., to an alpha-imino phosphate group of one of the present nucleotides). The bridging sulfur alternatively may be referred to as a phosphorothiolate. In examples where the bridging sulfur is coupled to the 5′ position of the sugar, the bridging sulfur may be referred to as a 5′-S-phosphorothiolate.

All ranges may include the upper and lower values, and all ranges and ratio limits disclosed herein may be combined. It is to be understood that unless specifically stated otherwise, references to “a,” “an,” and/or “the” may include one or more than one and that reference to an item in the singular may also include the item in the plural. Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item. Further, reference to, e.g., a “first” item and a “second” item does not mean that there are no intervening items, and such intervening items may be present.

Nucleotides with Alpha-Imino Phosphate Groups and Bridging Sulfurs, and Methods of Synthesizing the Same

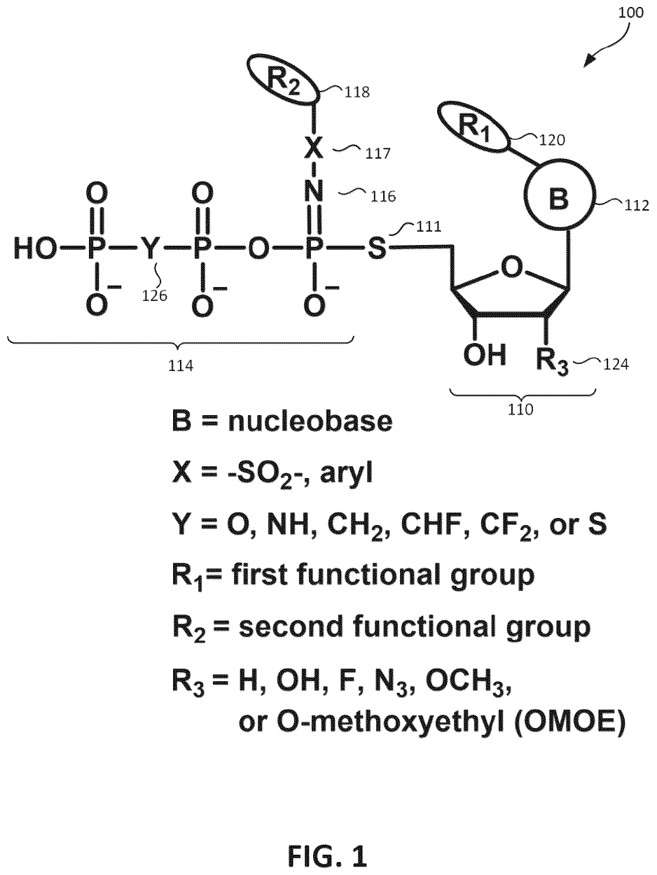

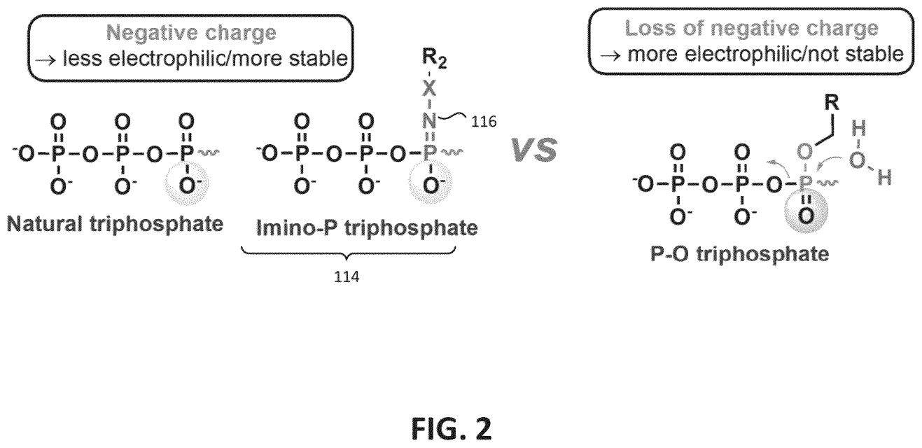

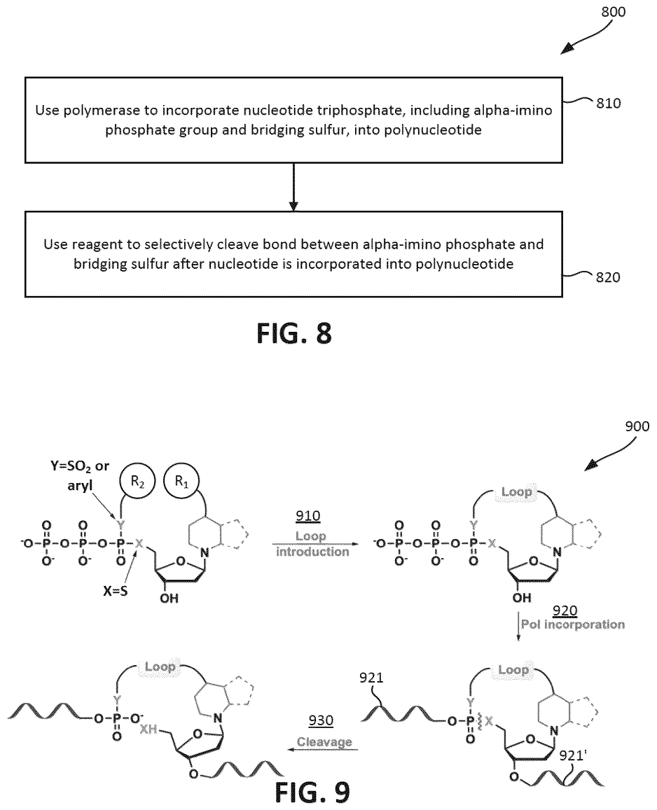

FIG. 1 illustrates example nucleotides provided herein. Referring now to FIG. 1, nucleotide 100 may include sugar 110, nucleobase 112 coupled to sugar 110, alpha-imino phosphate group 116, and sulfur atom 111 coupling the alpha-imino phosphate group 116 to sugar 110 (as such, sulfur atom 111 may be referred to as a “bridging” sulfur). Nucleotide 110 may include at least one phosphate group coupled to the alpha-imino phosphate group 116. Illustratively, alpha-imino phosphate group 116 may be part of triphosphate group 114. The alpha-imino phosphate group 116 may inhibit hydrolysis of triphosphate group 114 (or other phosphate group of which alpha-imino phosphate 116 is a member), and thus may enhance stability of nucleotide 100, for example as compared to nucleotides with an alpha-phosphate group that is coupled to a functional group using a moiety other than imino. For example, FIG. 2 schematically illustrates an example manner in which the alpha-imino phosphate group and bridging sulfur stabilize the present nucleotides, in which X represents sulfonyl or aryl. As shown in FIG. 2, like a natural triphosphate, the alpha-imino phosphate group 116 of triphosphate group 114 carries a negative charge which is stabilizes the triphosphate against hydrolysis. In comparison, an alpha P—O group of a triphosphate group does not carry a negative charge, is more electrophilic and thus less stable because it is more susceptible to hydrolysis of pyrophosphate. Moreover, the bond between the alpha-phosphorous and the bridging sulfur selectively may be cleaved, which may find practical application in a variety of contexts, such as described below with reference to FIGS. 8 and 9.

Referring again to the nonlimiting example illustrated in FIG. 1, nucleotide 100 further includes substituent 117 (also denoted X), which may be SO₂ or aryl, wherein the aryl optionally is substituted. In nonlimiting examples, the aryl is phenyl, and the phenyl is optionally substituted. Nonlimiting examples of substituents on the aryl (e.g., phenyl) include one or more halogens (such as fluorine), alkoxy, or amine. Nucleotide 100 optionally also includes functional group R₂ 118 coupled to the alpha-imino phosphate group 116 is via substituent 117. Nucleotide 100 optionally may include functional group R₁ 120 coupled to nucleobase 112. Functional group R₁ 120 may be of the same type as functional group R₂ 118, or may be of a different type than functional group R₂ 118. Optionally, functional group R₂ 118 may be coupled functional group R₁ 120 to form a loop. Nonlimiting examples of functional groups R₁ 120 and R₂ 118 are provided elsewhere herein.

In various examples, sugar 110 may include a five-carbon sugar. Illustratively, sugar 110 may include a natural sugar, such as ribose or deoxyribose. In other examples, sugar 110 may include a six carbon sugar, e.g., pyranose. Optionally, sugar 110 is a non-naturally occurring sugar, such as threose (as in threose nucleic acid, TNA). In various examples, sugar 110 may include an acyclic sugar moiety, e.g., may include glycerol (as in glycol nucleic acid, GNA). In various examples, sugar 110 may include a fluorine atom bound to the 2′ carbon of the sugar. Note that sugar 110 may be, but not necessarily be, cyclic. From the examples herein, it may be understood that sugar 110 illustratively may be a three-carbon sugar, a four-carbon sugar, a five-carbon sugar, or a six carbon sugar, and may be cyclic or acyclic. Nonlimiting examples of sugars, and associated backbones, are illustrated below, in which B represents the nucleobase:

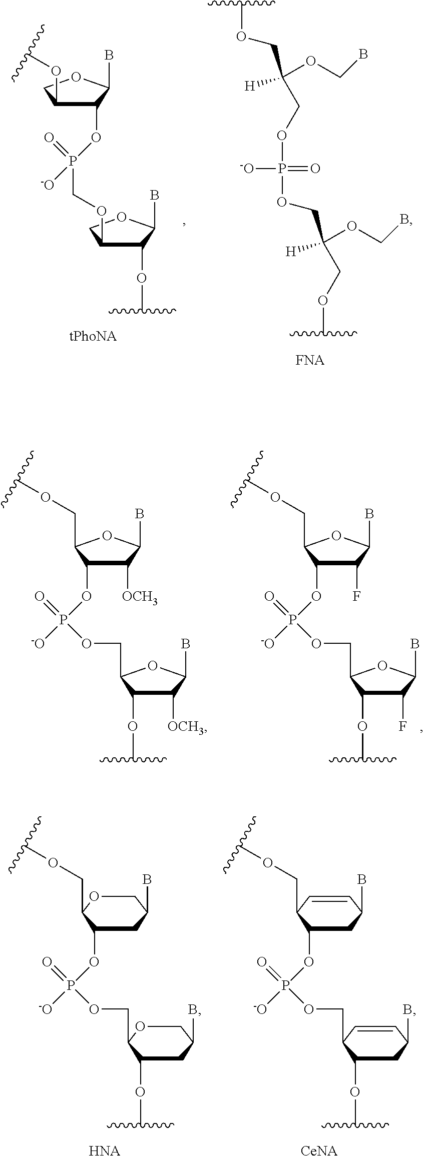

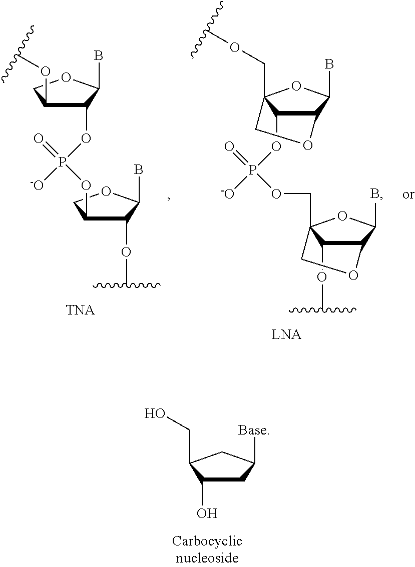

In various examples illustrated in FIG. 1, functional group R₁ 120 and/or functional group R₂ 118 may assist in the identification of nucleotide 100 in a sequencing application. For example, the reaction of functional group R₁ 120 and/or functional group R₂ 118 may assist in the identification of nucleotide 100. Functional group R₁ 120 and/or functional group R₂ 118 may include, illustratively, an amine, an azide, a carboxylic acid, a thiol, a tetrazine, a cyclooctyne, or an alkyne. Additionally, or alternatively, in some examples, functional group R₁ 120 and/or functional group R₂ 118 may include a fluorescent dye, an alkyl chain, or a polymer. Nonlimiting examples of polymers suitable for use in functional group R₁ 120 and/or functional group R₂ 118 include a synthetic peptide, a natural peptide, a synthetic polymer, a natural polymer, or a polynucleotide (e.g., a DNA polymer). Nonlimiting examples of a synthetic polymer include an ethylene glycol polymer, which may be referred to as poly(ethylene glycol), ethylene oxide polymer, polymethylene polymer, (ethylene oxide)phosphate polymer, and polymethylene phosphate polymer. In various examples in which functional group R₁ 120 and/or functional group R₂ 118 respectively includes a polymer, the polymer may include about 1 to about 50 repeating units, illustratively about 1 to about 10 repeating units, or about 11 to about 20 repeating units, or about 21 to about 50 repeating units.

In various examples, nucleotide 100 may include substituent 124 (R₃) which may be coupled to the 2′ carbon of sugar 110. Substituent 124 may act as a protecting group. Substituent 124 may be a protecting group for an RNA synthesis and/or a substituent such as fluorine atom or a methoxy group as an RNA analogue. Additionally, or alternatively, substituent 124 may be selected so as to increase the temperature at which a duplex including the nucleotide will dissociate. That is, the substituent 124 may increase the melting temperature (Tm) of a duplex including the nucleotide. Substituent 124 additionally, or alternatively, may improve resistance to a nuclease of a polynucleotide that includes the nucleotide 100. In non-limiting examples such as illustrated in FIG. 1, substituent 124 may include a H, OH, an azide, fluorine, a methoxy group, an ethoxy group, or an O-methoxyethyl (OMOE) group.

In various examples, nucleotide 100 may include substituent 126 which may be coupled between a beta phosphate and a gamma phosphate of triphosphate group 114. Without wishing to be bound by any theory, it is believed that substituent 126 may affect the stability of nucleotide 100. Illustratively, and without wishing to be bound by any theory, it is believed that the presence of a modification on the alpha phosphate may alter the electron density across the phosphate groups, resulting in instability, and that the substituent may potentially rebalance the electron density to regain sufficient stability of the triphosphate towards degradation (loss of phosphate groups). In nonlimiting examples such as illustrated in FIG. 1, substituent 126 may include an oxygen atom, an amine, a methylene, a fluoromethylene, a difluoromethylene, a sulfur atom, or a selenium atom.

It will be appreciated that a wide variety of nucleobases may be included in nucleotide 100. In examples in which nucleobase 112 is not coupled to functional group R₁ 120, nucleobase 112 may include a naturally occurring nucleobase such as a purine or pyrimidine, or may include a purine analog or pyrimidine analog. In examples in which nucleobase 112 is coupled to functional group R₁ 120, as illustrated in FIG. 1, nucleobase 112 may include a purine analog or pyrimidine analog to which the functional group is coupled.

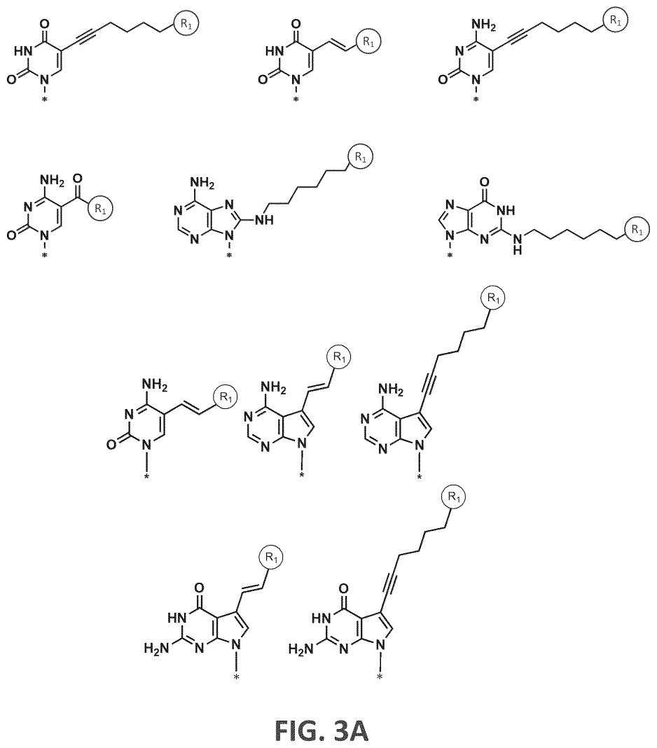

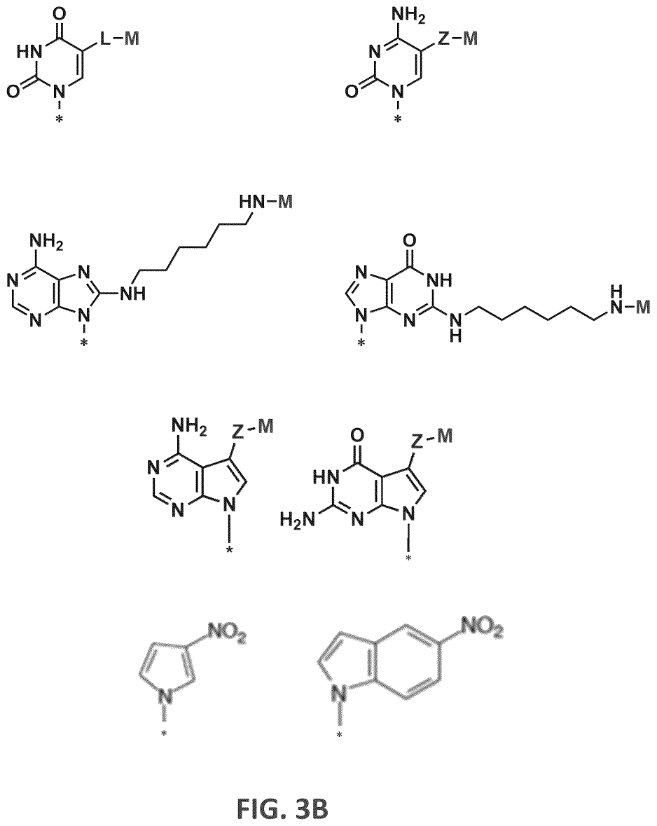

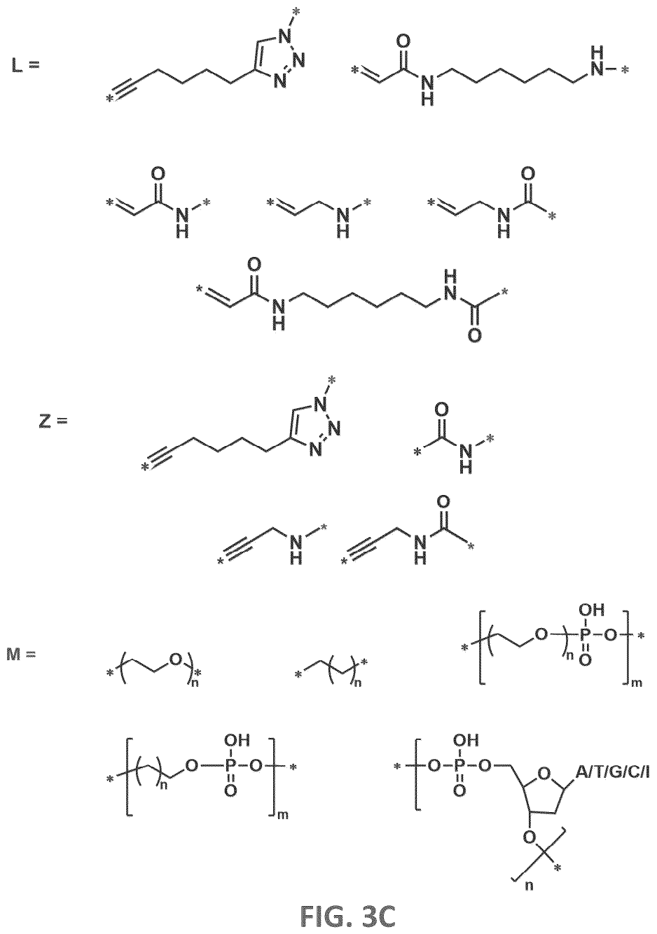

FIGS. 3A-3C schematically illustrate example modified nucleobases, and functional groups, that may be used in the present nucleotides. Nonlimiting examples of nucleobase 112 include adenine, cytosine, guanine, thymine, or uracil. Other nonlimiting examples of nucleobase 112 include 5-methylcytosine, 5-hydroxymethylcytosine, 5-formylcytosine, 5-carboxylcytosine, 4-methylcytosine, 6-methyladenine, 8-oxoguanine, or 8-oxoadenine. Still other nonlimiting examples of nucleobase 112 include inosine, xanthine, hypoxanthine, 2-aminoadenine, 6-methyl guanine, 4-thiouracil, 8-hydroxyl adenine or guanine, 7-methylguanine, 7-methyladenine, 8-azaguanine, 8-azaadenine, or the like. Still other nonlimiting examples of nucleobase 112 include may include isocytosine, isoguanine, 2-aminopurine, 2-propyl guanine, 2-propyl adenine, 2-thiouracil, 2-thiothymine, 2-thiocytosine, 5-thiouracil, 5-halocytosine, 5-propynyl uracil, 5-propynyl cytosine, 6-azo uracil, 6-azo cytosine, 6-azo thymine, 5-thiouracil, 8-halo adenine or guanine, 8-amino adenine or guanine, 8-thiol adenine or guanine, 8-thioalkyl adenine or guanine 5-halo substituted uracil or cytosine, 7-deazaguanine, 7-deazaadenine, 3-deazaguanine, 3-deazaadenine or the like.

In still other examples such as illustrated in FIG. 3A, nucleobase 112 may include a non-naturally occurring nucleobase, such as 5-(1,6-heptadiynyl) uracil, 5-(2-carboxyvinyl) uracil, 5-(1,6-heptadiynyl) cytosine, 5-carboxycytosine, 8-(1,6-diaminohexanyl) adenine, and 2-(1,6-diaminohexanyl) guanine, In still other examples such as illustrated in FIG. 3B, nucleobase 112 may include a non-naturally occurring nucleobase, such as 5-(7-(1,2,3-triazole) hept-1-ynyl) uracil, 5-(N-(6-aminohexyl) acrylamide) uracil, 5-(methylacrylamido) uracil, 5-(N-allylmethylamino) uracil, 5-(N-allylacetamidyl) uracil, 5-(7-(1,2,3-triazole) hept-1-ynyl) cytosine, and 5-(methylacetamido) cytosine, 8-(1,6-diaminohexanyl) adenine, or 2-(1,6-diaminohexanyl) guanine. As illustrated in FIG. 3C, nucleobase 112 of FIG. 3B may in some examples include an ethylene glycol polymer, which may be referred to as poly(ethylene glycol), ethylene oxide polymer, polymethylene polymer, (ethylene oxide)phosphate polymer, polymethylene phosphate polymer, or polynucleotide (e.g., DNA polymer). As illustrated in FIG. 3B, non-naturally occurring nucleobases suitable for use in some examples herein may include “universal” bases.

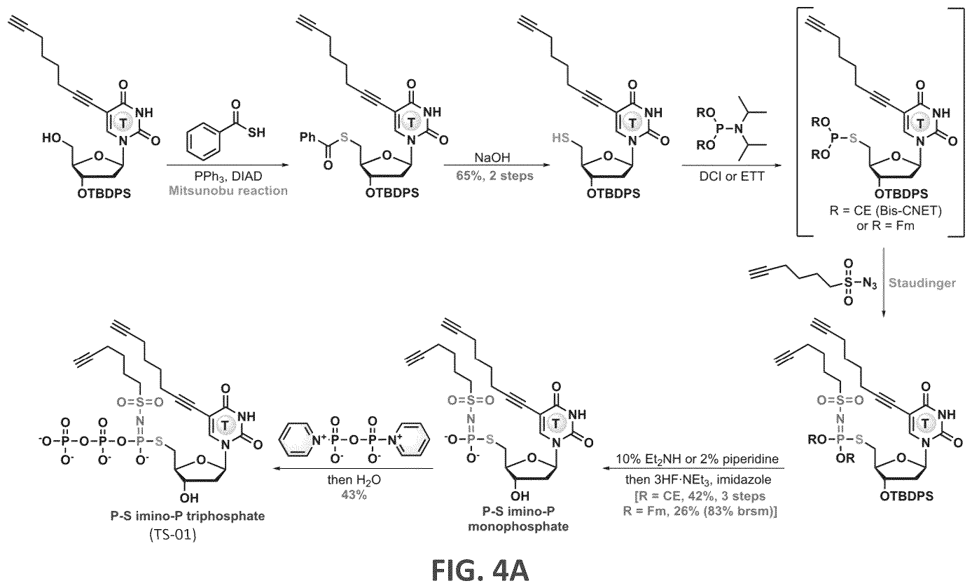

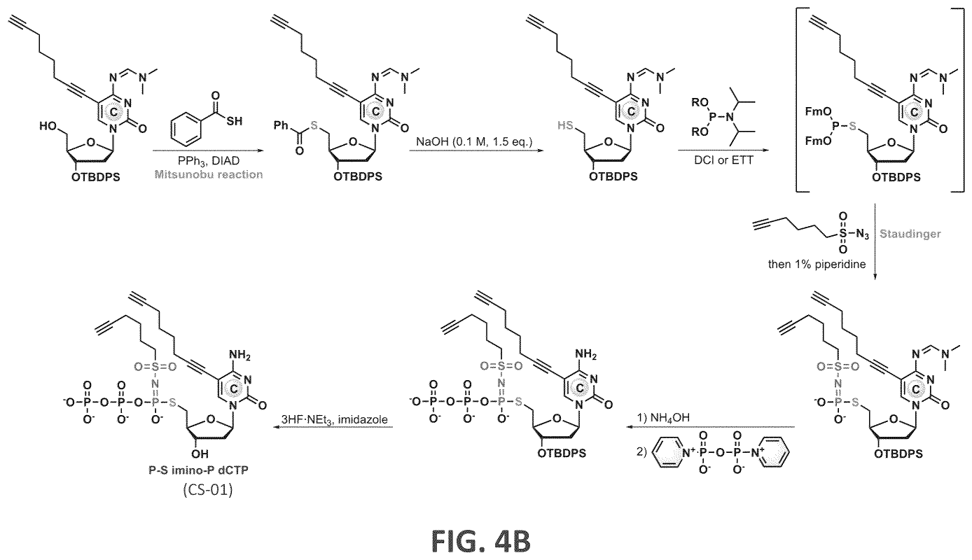

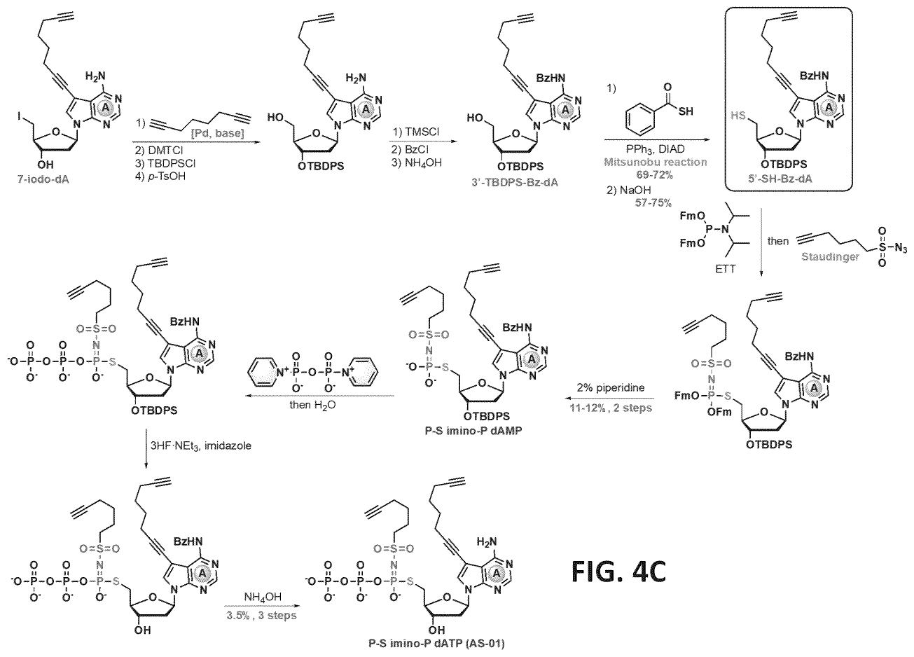

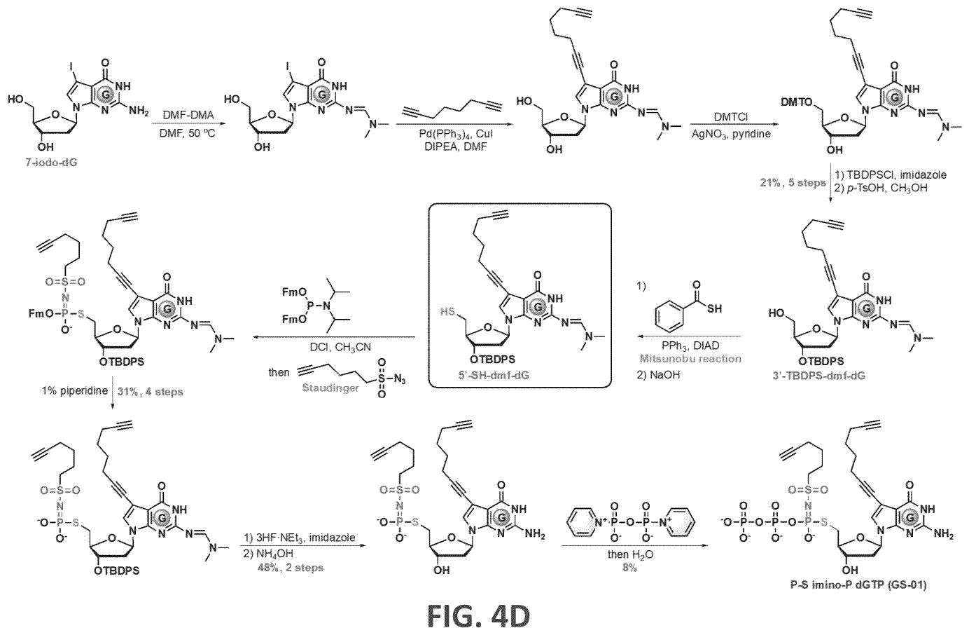

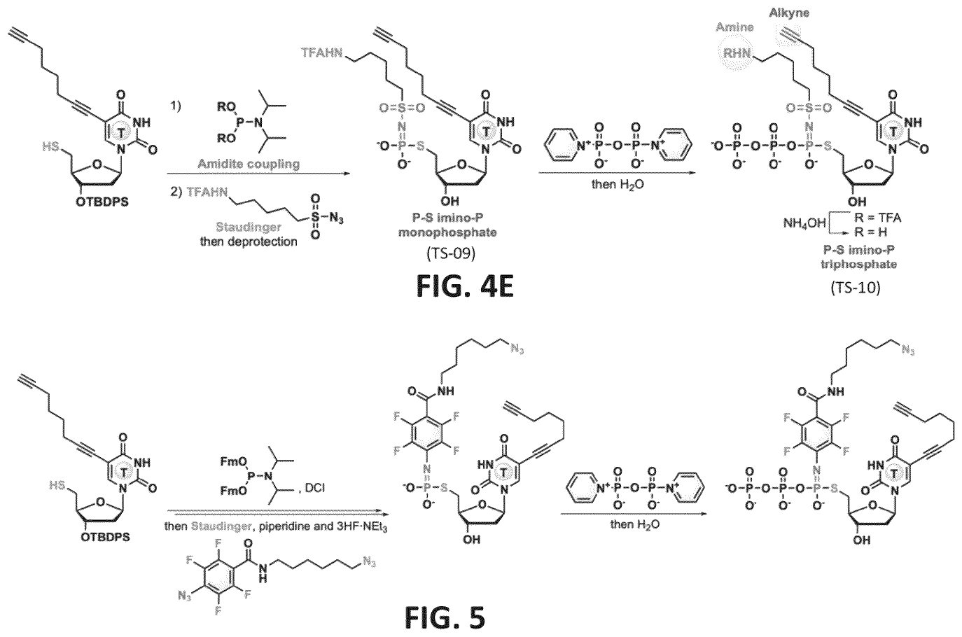

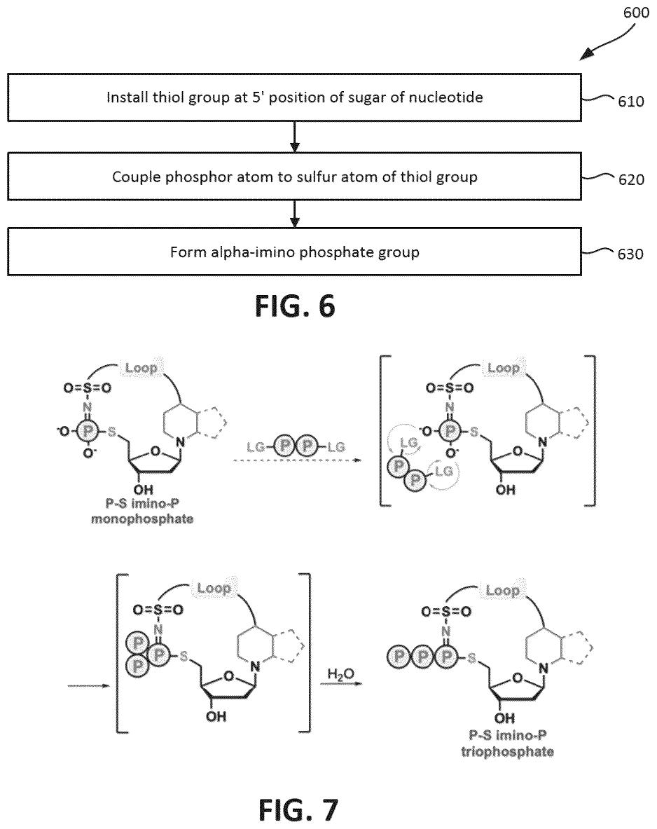

Nucleotides such as described with reference to FIGS. 1, 2, and 3A-3C may be synthesized in any suitable manner. For example, FIG. 6 illustrates a flow of operations in an example method of synthesizing a nucleotide with an alpha-imino phosphate group and a bridging sulfur. As illustrated in FIG. 6, method 600 may include installing a thiol group at a 5′ position of a sugar of the nucleotide (operation 610). Method 600 also may include coupling a phosphor atom to the sulfur atom of the thiol group (operation 620). Operation 600 also may include coupling, to the phosphor atom, an imino group coupled to a sulfonyl group or an aryl group (operation 630). In a manner such as now will be described with reference to FIGS. 4A-4E, installing the thiol group optionally may include using a Mitsunobu reaction followed by reduction; coupling the phosphor atom to the sulfur atom may include using a phosphoramidite; and/or coupling the imino group to the phosphor atom comprises using a Staudinger reaction.

For example, FIG. 4A schematically illustrates an example method of synthesizing deoxythymidine with a sulfonyl group coupled to the alpha-imino phosphate group and a 5′-S-phosphorothiolate bridging sulfur. The 5′-OH precursor may be commercially obtained or suitably prepared, and may include a suitable protective group (such as tert-butyldiphenylsilyl (TBDPS)) at the sugar's 3′-O position. The precursor may be subjected to the Mitsunobu reaction (BzSH, DIAD, PPh₃) followed by hydrolysis of the benzoyl group (e.g., using NaOH) to install the thiol group at the 5′ position of the sugar (operation 610). The thiol group may be coupled to a phosphoramidite (operation 620), such as Fm (fluorenylmethoxycarbonyl) amidite, and an activator such as DCI (4,5-dicyanoimidazole) or ETT (5-(Ethylthio)-1H-tetrazole) in a suitable solvent such as acetonitrile. The P(III) intermediate then may be oxidized, such as with hexynyl sulfonyl azide under Staudinger reaction conditions to form the alpha-imino phosphate group (operation 630). The Fm groups (or other protective groups of the phosphoramidite) may be selectively removed, such as with 5% piperidine in acetonitrile; and the protective group at the 3′-O position may be selectively removed, such as with HF-Et₃N, to obtain the monophosphate nucleoside. Subsequent phosphorylation with activated pyrophosphate followed by hydrolysis with water or buffer (e.g., triethylammonium acetate (TEAA)) may be used to obtain the final triphosphate (here, referred to as TS-01).

FIG. 4B schematically illustrates another example method of synthesizing deoxycytidine with a sulfonyl group coupled to the alpha-imino phosphate group and a 5′-S-phosphorothiolate bridging sulfur. The 5′-OH precursor may be commercially obtained or suitably prepared, which includes a protective group at the nucleobase's amine. Similarly as described with reference to FIG. 4A, the Mitsunobu reaction followed by hydrolysis of the benzoyl group may be used to install the thiol group at the 5′ position of the sugar (operation 610). The thiol group may be coupled to a phosphoramidite (operation 620) using a suitable activator. The P(III) intermediate then may be oxidized under Staudinger reaction conditions to form the alpha-imino phosphate group (operation 630). The groups protecting the phosphoramidite may be selectively removed, the protective group at the 3′-O position may be selectively removed, and the protective group at the nucleobase's amine may be removed, to obtain the monophosphate nucleoside. Subsequent phosphorylation with activated pyrophosphate followed by hydrolysis with water or buffer may be used to obtain the final triphosphate (here, referred to as CS-01).

FIG. 4C schematically illustrates another example method of synthesizing deoxyadenosine with a sulfonyl group coupled to the alpha-imino phosphate group and a bridging sulfur. The 5′-OH precursor may be commercially obtained or suitably prepared. In this nonlimiting example, Sonogashira coupling (Pd(Ph₃)₄, Cul, DIPEA, DMF) may be used to install the functional group at the nucleobase, the sugar's 3′-O position is protected using a suitable protective group, and which the nucleobase's amine is protected using a suitable protective group. Similarly as described with reference to FIG. 4A, the Mitsunobu reaction followed by hydrolysis of the benzoyl group may be used to install the thiol group at the 5′ position of the sugar (operation 610). The thiol group may be coupled to a phosphoramidite (operation 620) using a suitable activator. The P(III) intermediate then may be oxidized under Staudinger reaction conditions to form the alpha-imino phosphate group (operation 630). The groups installed protecting the phosphoramidite may be selectively removed, and the protective group at the 3′-O position may be selectively removed. Subsequent phosphorylation with activated pyrophosphate followed by hydrolysis with water or buffer, and removal of the protective group at the nucleobase's amine, may be used to obtain the final triphosphate (here, referred to as AS-01).

FIG. 4D schematically illustrates another example method of synthesizing deoxyguanosine with a sulfonyl group coupled to the alpha-imino phosphate group and a bridging sulfur. The 5′-OH precursor may be commercially obtained or suitably prepared. In this nonlimiting example, Sonogashira coupling may be used to install the functional group at the nucleobase, and the sugar's 3′-O position is protected using a suitable protective group. Similarly as described with reference to FIG. 4A, the Mitsunobu reaction followed by hydrolysis of the benzoyl group may be used to install the thiol group at the 5′ position of the sugar (operation 610). The thiol group may be coupled to a phosphoramidite (operation 620) using a suitable activator. The P(III) intermediate then may be oxidized under Staudinger reaction conditions to form the alpha-imino phosphate group (operation 630). The protective groups of the phosphoramidite may be selectively removed, and the protective group at the 3′-O position may be selectively removed, to obtain the monophosphate nucleoside. Subsequent phosphorylation with activated pyrophosphate followed by hydrolysis may be used to obtain the final triphosphate (here, referred to as GS-01).

While the nucleobases illustrated in FIGS. 4A-4D are coupled to a particular functional group including an alkyne, it should be appreciated that other functional groups suitably may be used. Additionally, while the alpha-imino phosphate illustrated in FIGS. 4A-4D is coupled to a particular functional group including an alkyne, it should be appreciated that other functional groups suitably may be used. The functional groups coupled to the nucleobases and the alpha-imino phosphates may be the same as one another, or may be different than one another. For example, FIG. 4E illustrates another example method of synthesizing a nucleotide with a sulfonyl group coupled to the alpha-imino phosphate group and a bridging sulfur. In this example, the Staudinger reaction is used to introduce a different functional group at the alpha-imino phosphate than is installed at the nucleobase. More specifically, a thiol is installed at the sugar's 5′ position (operation 610), and then coupled to a phosphoramidite (operation 620), in a manner such as described with reference to FIGS. 4A-4E. The Staudinger reaction then is used to introduce a protected amine group, such as TFAHN (trifluoroacetylamino), coupled to the alpha-imino phosphate group (operation 630). Subsequent phosphorylation with activated pyrophosphate, followed by hydrolysis, generates the triphosphate with protected amine (referred to in FIG. 4E as TS-09). Ammonium hydroxide, or other suitable reactant, then is used to reduce the protected amine group to an amine and generate the final triphosphate (referred to in FIG. 4E as TS-10). Still other strategies readily may be developed based on the present teachings to respectively install other suitable functional groups at the nucleobase and alpha-imino phosphate.

Additionally, while FIGS. 4A-4E illustrate nonlimiting examples in which the alpha-imino phosphate is coupled to an SO₂ group, the schemes readily may be modified to install an aryl group instead of an SO₂ group. For example, FIG. 5 schematically illustrates an example method of synthesizing deoxythymidine with a phenyl group coupled to the alpha-imino phosphate group and a bridging sulfur. In this example, a thiol is installed at the sugar's 5′ position (operation 610) in a manner such as described with reference to FIGS. 4A-4E, and then coupled to a phosphoramidite (operation 620), in a manner such as described with reference to FIGS. 4A-4E. The Staudinger reaction then is used to install an aryl (e.g., phenyl, illustratively a fluorinated phenyl) group at the alpha-imino phosphate (operation 630). Note that for Staudinger reactions, ideal partners are electron-rich phosphorous with electron-poor azide (like the sulfonyl azide we also used). The aliphatic azide is more electron-rich than the azide attached to an electron-deficient tetrafluorophenyl ring, so the latter reacts faster with the P(III) intermediate. As illustrated here, the aryl (e.g., phenyl) group is coupled to a functional group corresponding to R₂ in FIG. 1. Subsequent phosphorylation with activated pyrophosphate followed by hydrolysis may be used to obtain the final triphosphate. Aryl groups other than phenyl may be installed similarly.

It will be apparent from the present teachings that a wide variety of functional groups may be installed using methods such as described with reference to FIGS. 4A-4E, 5, and 6. In some examples, the sulfonyl group or the aryl group of the nucleotide is coupled to a first functional group, and the nucleobase is coupled to a second functional group. Optionally, the first functional group may be coupled to the second functional group to form a loop. The loop may be formed either before or after synthesis of the nucleotide is complete. For example, FIG. 7 illustrates a flow of operations in an example method of coupling a pyrophosphate group to an alpha-imino phosphate group. Here, the first and second functional groups are coupled to one another to form a loop. Subsequent phosphorylation with activated pyrophosphate followed by hydrolysis may be used to obtain the final triphosphate. While FIG. 7 illustrates the alpha-imino phosphate coupled to a sulfonyl group, in other examples the alpha-imino phosphate may be coupled to an aryl group.

The present nucleotides may be used in any suitable manner or application. For example, FIG. 8 illustrates a flow chart of operations in an example method of using a nucleotide with an alpha-imino phosphate group and a bridging sulfur. Method 800 illustrated in FIG. 8 includes using a polymerase to incorporate a nucleotide, including an alpha-imino phosphate group and a bridging sulfur, into a polynucleotide (operation 810). For example, any suitable polymerase may add any of the present nucleotides, nonlimiting examples of which are described with reference to FIGS. 1-7, to a growing polynucleotide based on the sequence of a template polynucleotide. Method 800 illustrated in FIG. 8 also includes using a reagent to selectively cleave the bond between the alpha-imino phosphate and bridging sulfur after the nucleotide is incorporated into the polynucleotide (operation 820). Nonlimiting examples of reagents which may selectively cleave such bond, e.g., which may not cleave any other bond in the nucleotide besides the bond between the alpha-imino phosphate and bridging sulfur, include Ag(I) metal salts (e.g., AgNO₃); Au(III) metal salts (e.g., AuCl₃); Pd(II) metal salts (e.g., Na₂PdCl₄); and Oxone (2KHSO₅·KHSO₄·K₂SO₄).

In some examples, a plurality of the present nucleotides (e.g., two or more, three or more, five or more, or ten or more) are incorporated into the polynucleotide, based on the sequence of a template polynucleotide. The polynucleotide then is contacted with the reagent, and the reagent cleaves the bond between the alpha-imino phosphate and bridging sulfur in each nucleotide of the plurality.

Operations such as described with reference to FIG. 8 may be incorporated into any suitable practical application. FIG. 9 schematically illustrates operations in an example method 900 for using a nucleotide with an alpha-imino phosphate group and a bridging sulfur. Method 900 illustrated in FIG. 9 includes introducing a loop into the nucleotide (operation 910). For example, as illustrated in FIG. 9, the nucleotide may include functional group R₁ coupled to the nucleobase (e.g., purine or pyrimidine) and functional group R₂ coupled to the alpha-imino phosphate group, in a manner such as described with reference to FIGS. 1-7. The functional groups R₁ and R₂ may be coupled together to form a loop. The resulting nucleotide may be referred to as a “cyclic loop nucleotide” or “CLN”. Method 900 illustrated in FIG. 9 also includes using a suitable polymerase to incorporate the CLN into a growing polynucleotide 921, based on the sequence of a template polynucleotide to which polynucleotide 921 is substantially complementary (operation 920). Method 900 illustrated in FIG. 9 also includes cleaving the bond between the alpha-imino phosphate group and the bridging sulfur (bond indicated with squiggle in FIG. 9) in the CLN, using a suitable reagent (operation 930). In the nonlimiting example illustrated in FIG. 9, such cleavage is performed after having incorporated one or more (e.g., two or more, or five or more) additional CLNs to further extend the growing polynucleotide 921 (subsequently added portion denoted 921′ in FIG. 9). However, it will be appreciated that such cleavage may be performed at any suitable time, e.g., after incorporating just a single CLN.

Note that any of the operations described with reference to FIG. 9 may be independently performed at different times than one another, and by different entities than one other. For example, a first entity (such as a nucleotide manufacturer) may introduce the loop into the nucleotide (operation 910) to form the CLN, before or after generating the triphosphate in a manner such as described with reference to FIG. 7. A second entity (such as an entity who wishes to sequence a template polynucleotide) may use a polymerase to incorporate the CLN into nucleotide 921 (operation 920) and may use a reagent to cleave the bond between the alpha-imino phosphate group and the bridging sulfur (operation 930).

Also provided herein are kits for performing operations such as described herein. For example, a kit may include a nucleotide an alpha-imino phosphate group and a bridging sulfur, and a reagent for selectively cleaving the bond between the sulfur atom and the alpha-imino phosphate. The reagent may include an Ag(I) metal salt, Au(III) metal salt, Pd(II) metal salt, or Oxone, or other suitable reagent. The kit may include a plurality of such nucleotides, and a sufficient quantity of the reagent to cleave the bonds between the sulfur atom and the alpha-imino phosphate in such nucleotides. In some examples, the kit further includes a polymerase for incorporating the nucleotide(s) into a polynucleotide.

### Working Examples

The following examples are purely illustrative, and not limiting of the present invention.

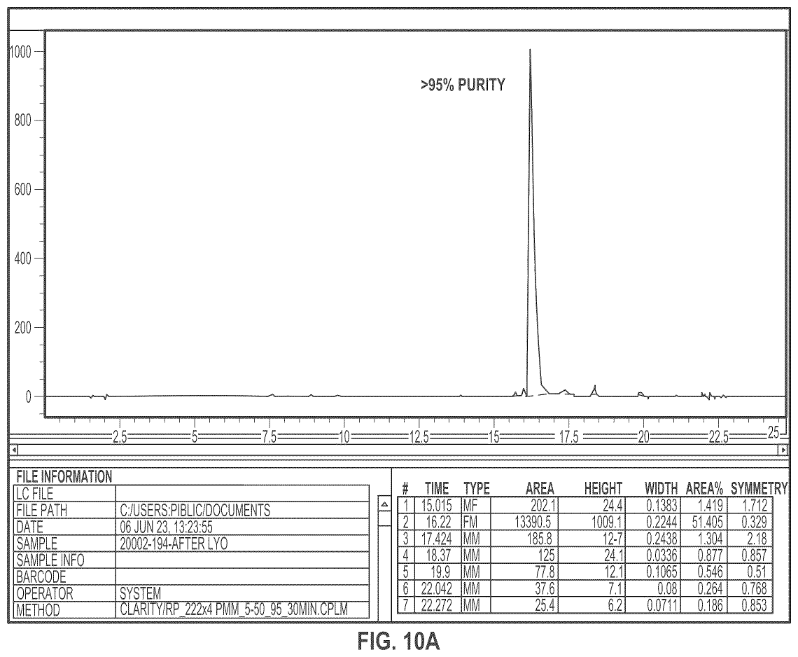

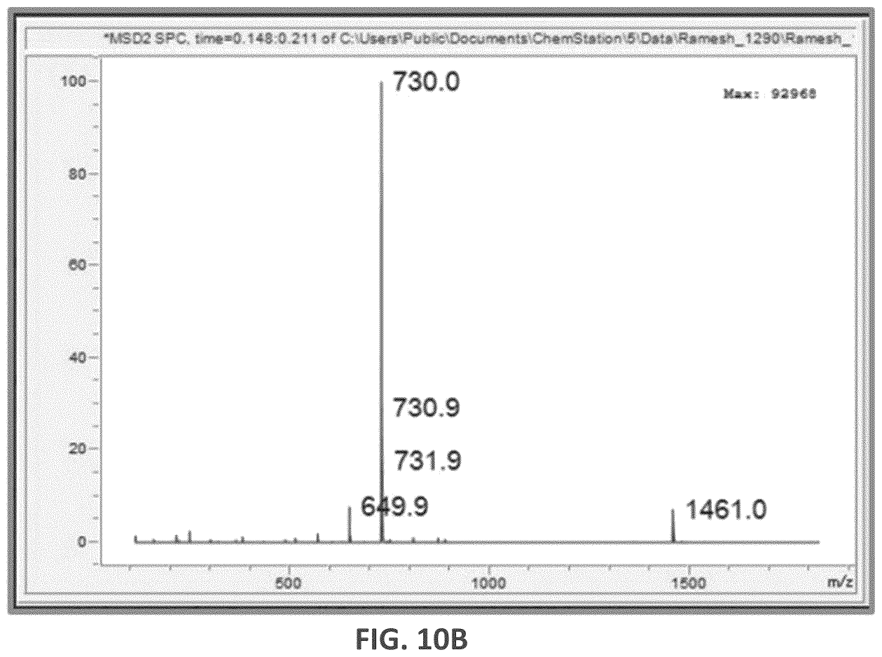

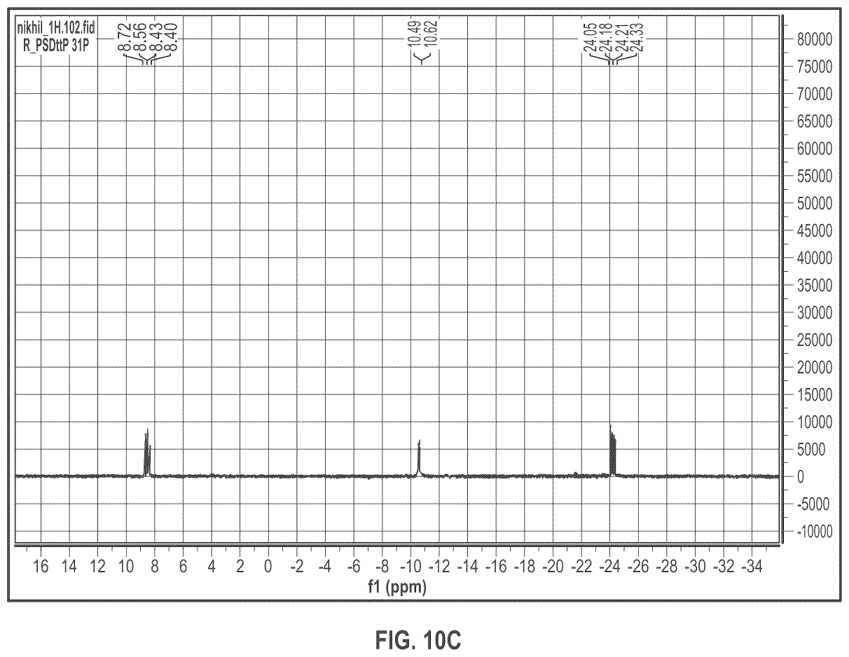

FIGS. 10A-10C respectively illustrate mass spectrometry (MS), high-performance liquid chromatography (HPLC), and ³¹P nuclear magnetic resonance (NMR) data for deoxythymidine synthesized in accordance with FIG. 4A (TS-01). The MS data shows the peak with the expected mass of the triphosphates, showing that the desired product was obtained. The HPLC data show the high purity of the triphosphates obtained. The ³¹P NMR data show the typical NMR peaks of a triphosphate (performed only for TS-01).

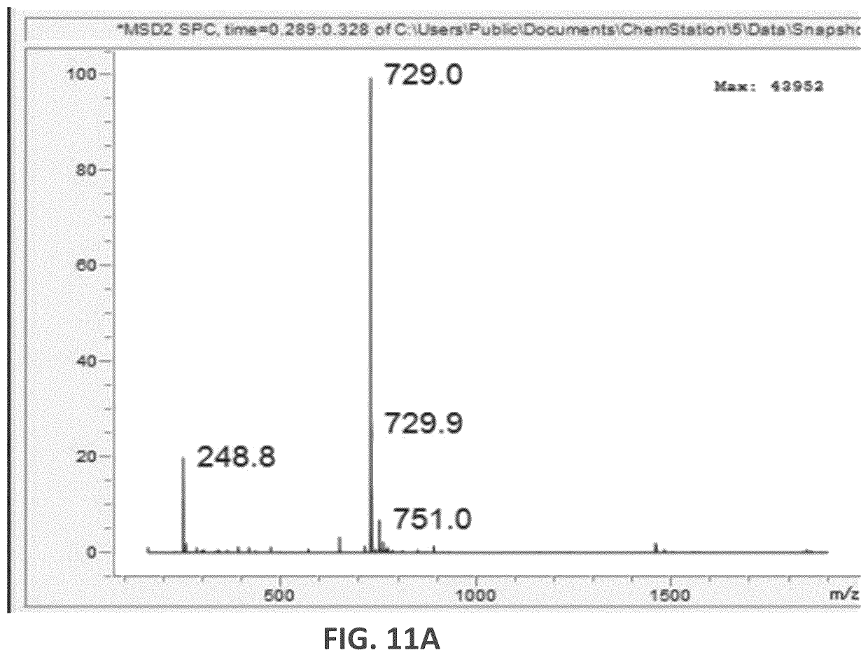

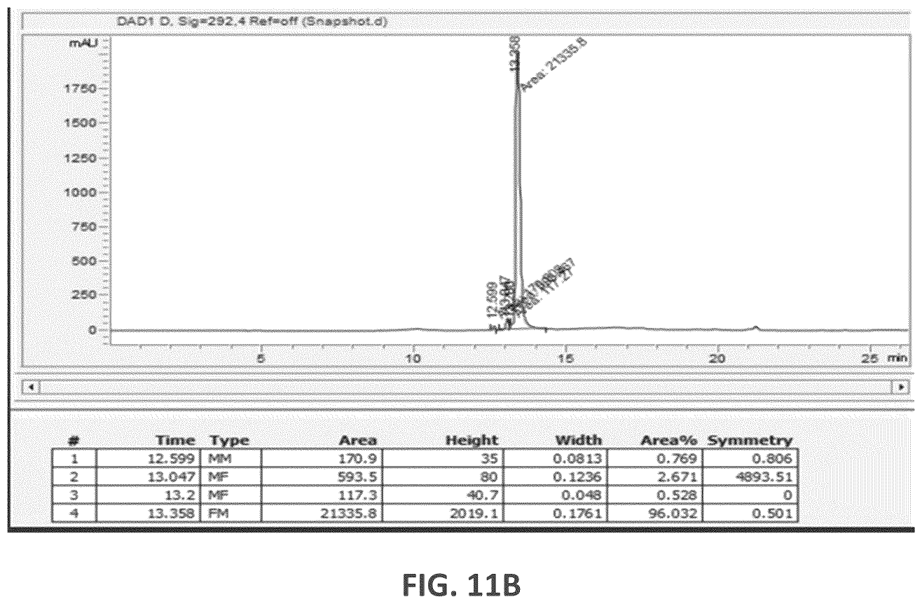

FIGS. 11A-11B respectively illustrate MS and HPLC data for deoxycytidine synthesized in accordance with FIG. 4B (CS-01). The MS data shows the peak with the expected mass of the triphosphates, showing that the desired product was obtained. The HPLC data show the high purity of the triphosphates obtained.

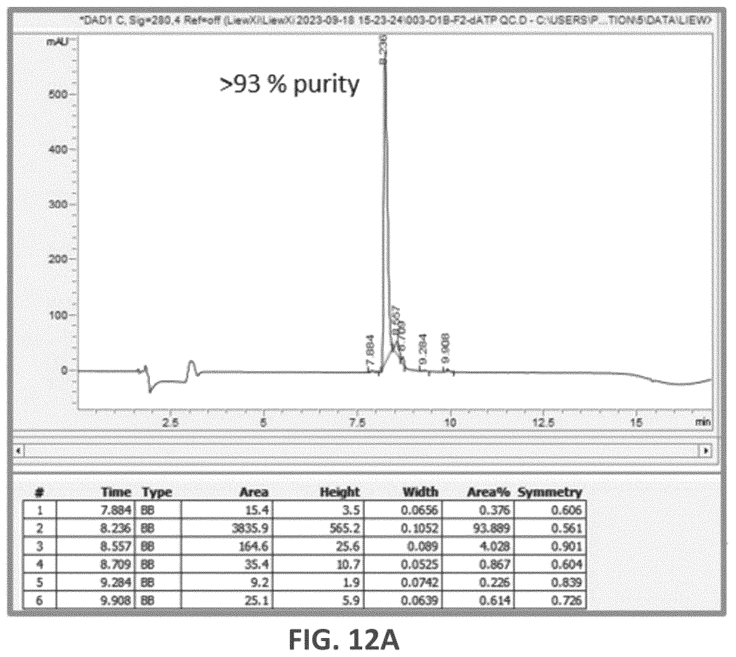

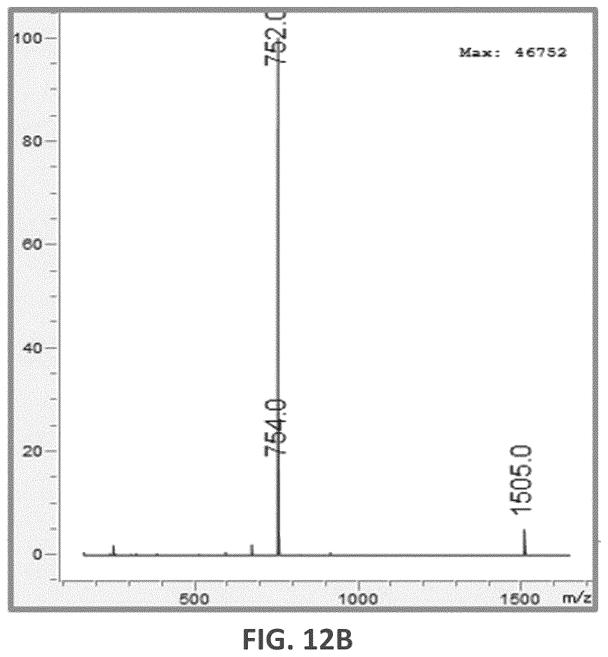

FIGS. 12A-12B respectively illustrate MS and HPLC data for deoxyadenosine synthesized in accordance with FIG. 4C (AS-01). The MS data shows the peak with the expected mass of the triphosphates, showing that the desired product was obtained. The HPLC data show the high purity of the triphosphates obtained.

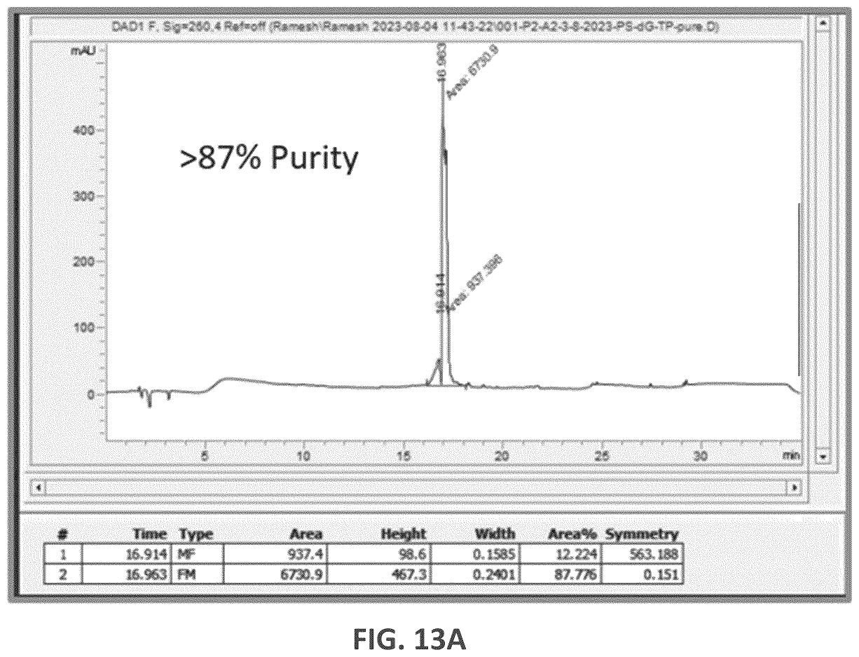

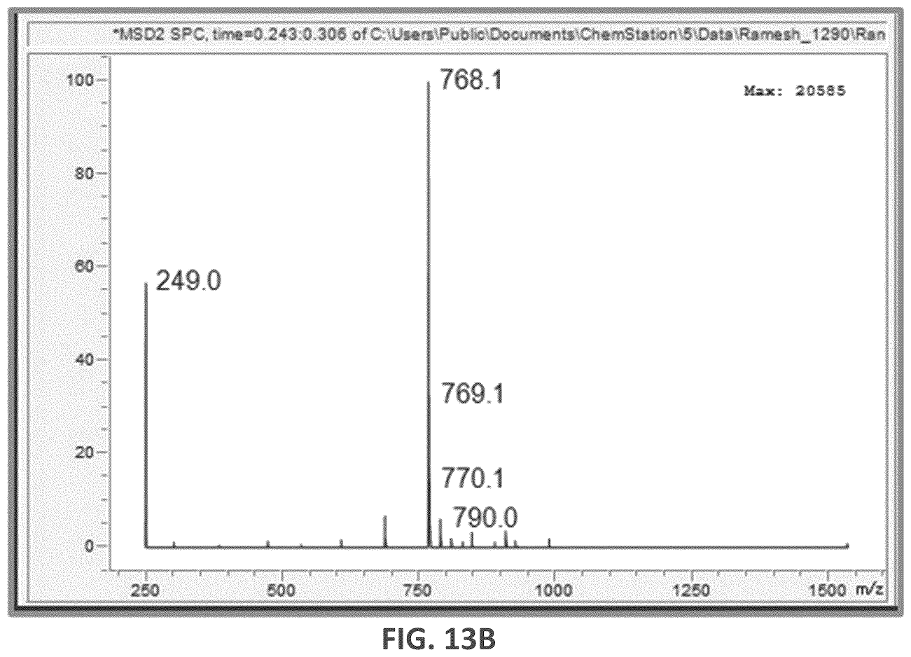

FIGS. 13A-13B respectively illustrate MS and HPLC data for deoxyguanosine synthesized in accordance with FIG. 4D (GS-01). The MS data shows the peak with the expected mass of the triphosphates, showing that the desired product was obtained. The HPLC data show the high purity of the triphosphates obtained.

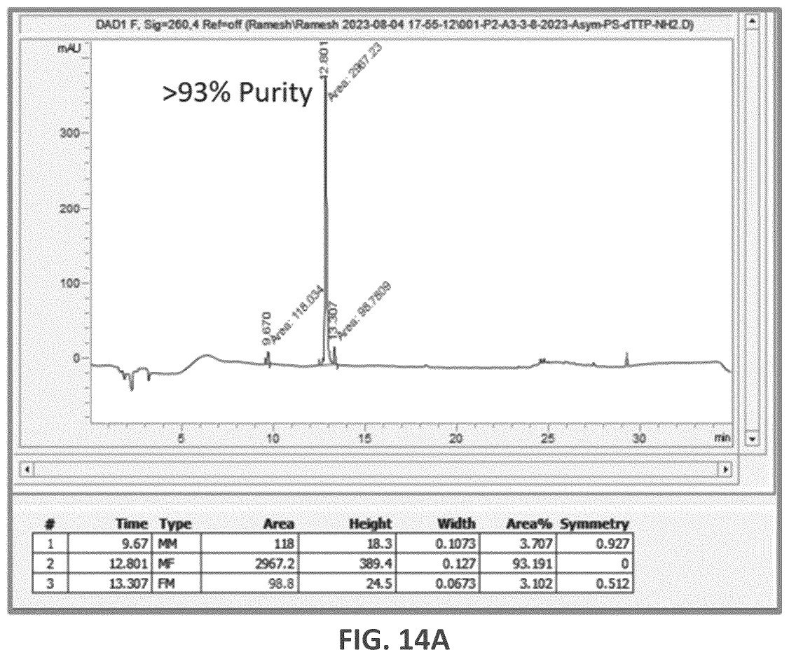

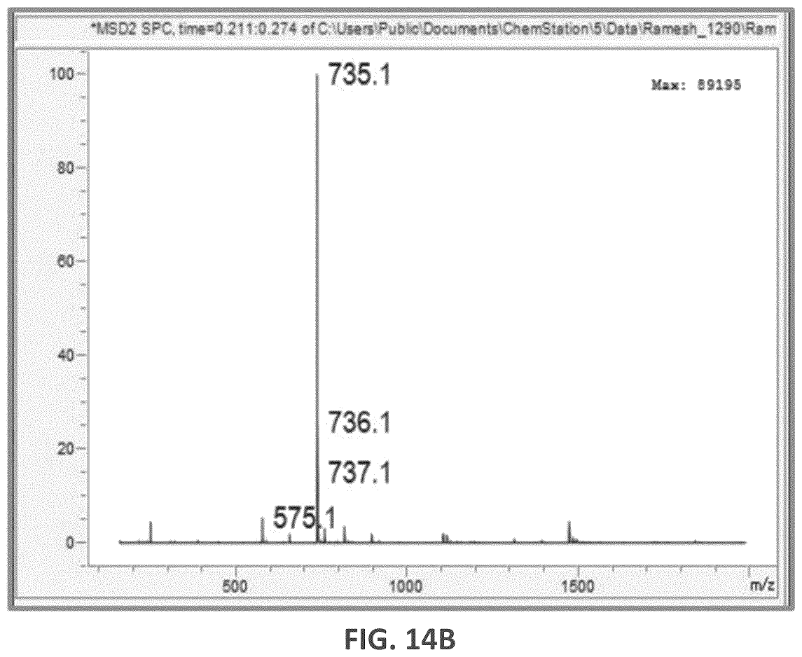

FIGS. 14A-14B respectively illustrate MS and HPLC data for deoxythymidine synthesized in accordance with FIG. 4E (TS-10). The MS data shows the peak with the expected mass of the triphosphates, showing that the desired product was obtained. The HPLC data show the high purity of the triphosphates obtained.

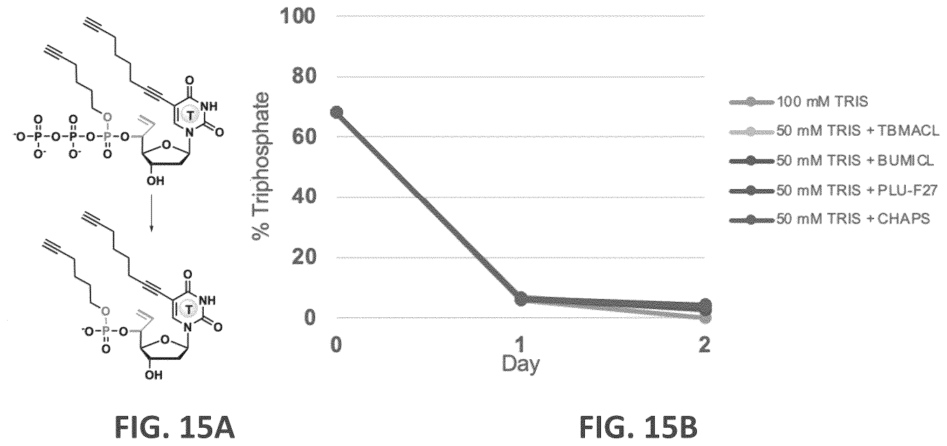

In a manner such as described further above, e.g., with reference to FIG. 2, the present alpha-imino phosphate groups provide significant stability advantages against hydrolysis of the triphosphate group as compared to nucleotides which lack alpha-imino phosphate groups. For example, FIG. 15A schematically illustrates hydrolysis of an example nucleotide triphosphate lacking an alpha-imino phosphate group and bridging sulfur. FIG. 15B schematically illustrates the percent triphosphate as a function of time at room temperature, for the example nucleotide triphosphate of FIG. 15A. The different data points illustrated in FIG. 15B were obtained using different buffer solutions that were used to attempt to stabilize the triphosphate. The staging conditions were as follows: 30 μM nucleotide, 30 mM TBMACl or BUMICl, 2% surfactant (PLU-F27 or CHAPS), and 50/100 mM Tris, pH 7.5, 20° C. The nucleotide triphosphate was analyzed using Triart C18 1.9 μm 150×3 mm column, flow rate of 0.5 mL/min, eluted with a mixture of 0.25 mM TEAB pH 7.2 and ACN at 40° C. It may be seen from FIG. 15B that after one day, less than about 10% of the triphosphate groups remained, and after two days even fewer of the triphosphate groups remained.

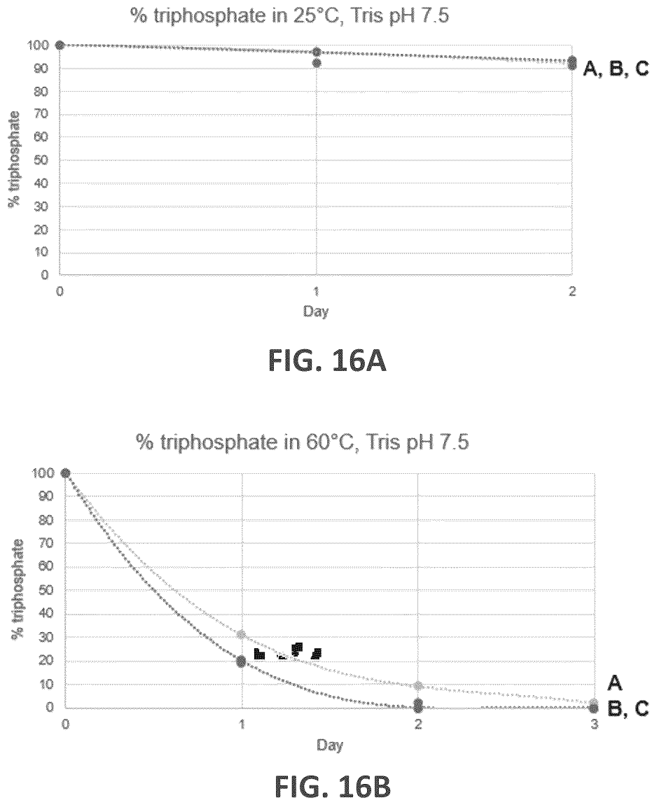

In comparison, FIG. 16A illustrates the percent triphosphate as a function of time, at room temperature, for example nucleotide triphosphates including an alpha-imino phosphate group and bridging sulfur, and FIG. 16B illustrates the percent triphosphate as a function of time, at elevated temperature, for example nucleotide triphosphates including an alpha-imino phosphate group and bridging sulfur. In FIGS. 16A-16B, A corresponds to data from TS-01 prepared in accordance with FIG. 4A, B corresponds to data from TS-09 prepared in accordance with FIG. 4E (before deprotecting the amine) and C corresponds to data from TS-10 prepared in accordance with FIG. 4E (after deprotecting the amine), structures shown again below:

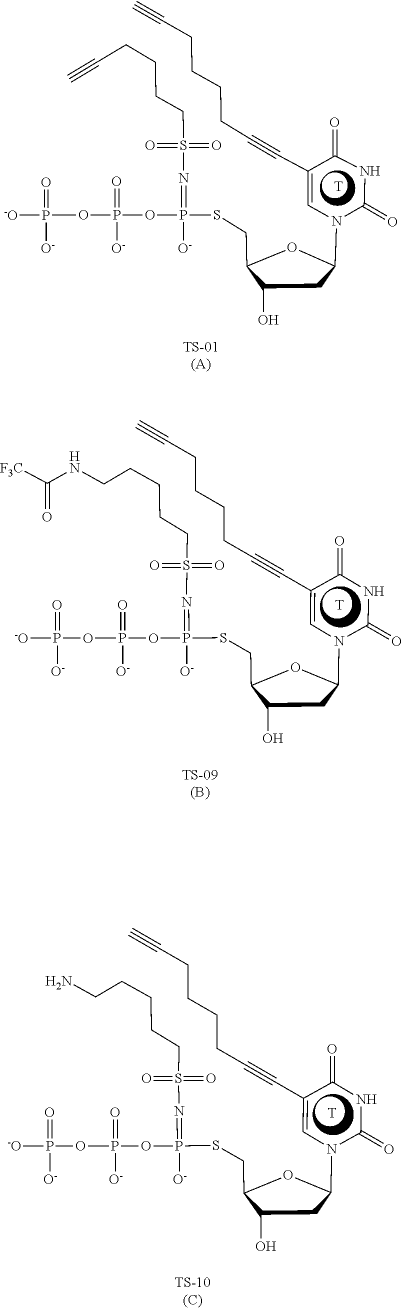

From FIG. 16A, it may be seen that even after two days, over 90% of triphosphate groups remained at room temperature. From FIG. 16B, it may be seen that the percent triphosphate decreased more rapidly at elevated temperature, after one day and two days there was still significantly more triphosphate present than for the nucleotide triphosphate of FIG. 15A, and the triphosphates were fully hydrolyzed after 3 days at 60° C. Accordingly, from FIGS. 16A-16B, it may be understood that the alpha-imino phosphate groups significantly inhibit hydrolysis of nucleotides' triphosphate groups, e.g., as compared to nucleotides which lack alpha-imino phosphate groups. Accordingly, the alpha-imino phosphate groups are expected to significantly extend the stability of the nucleotide triphosphates.

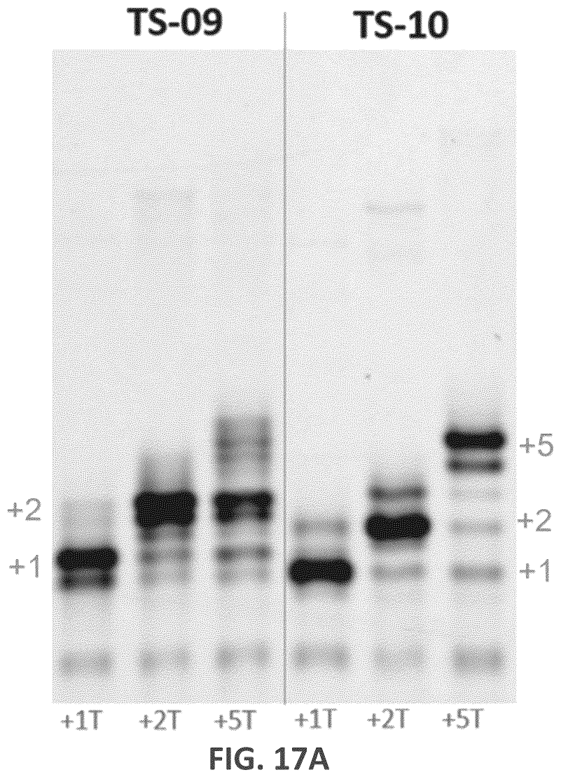

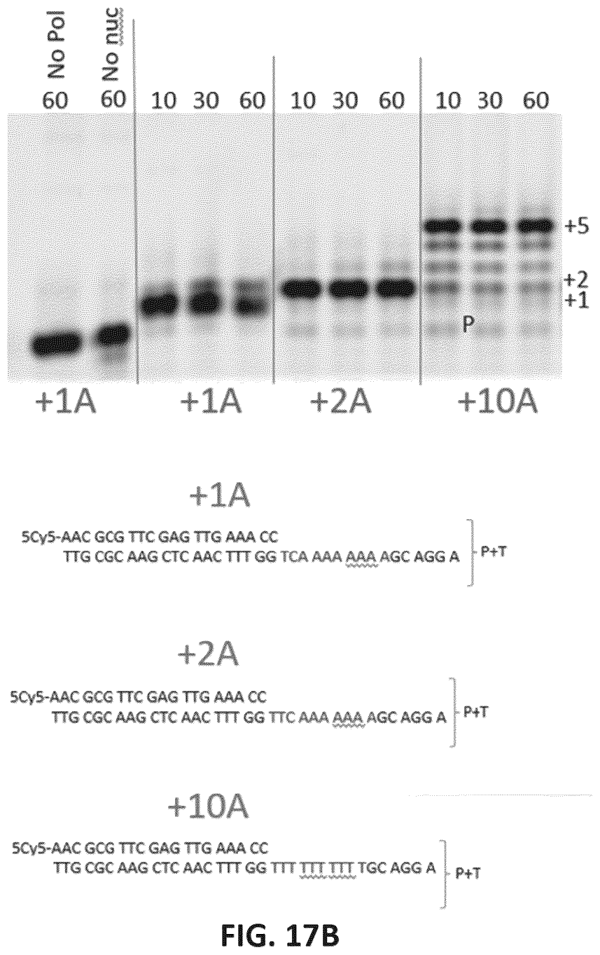

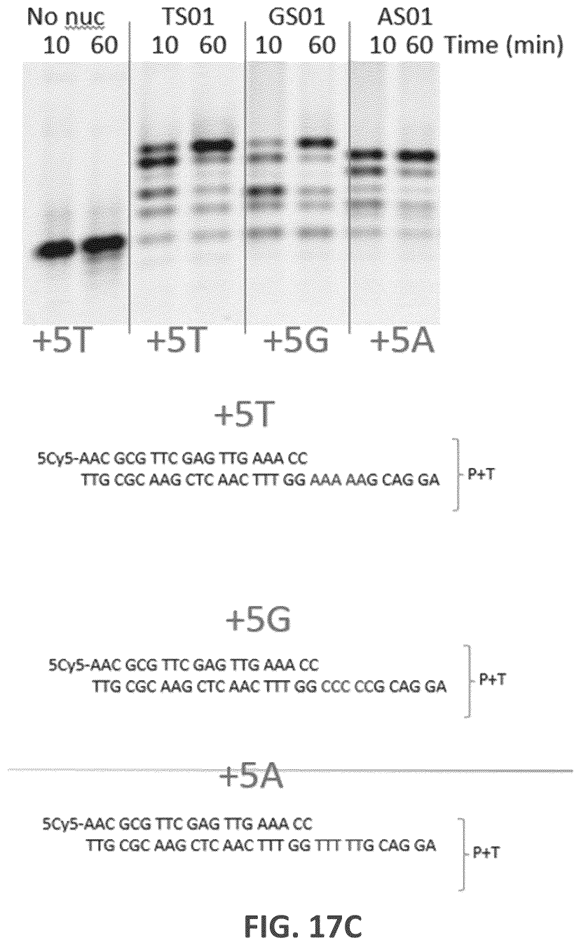

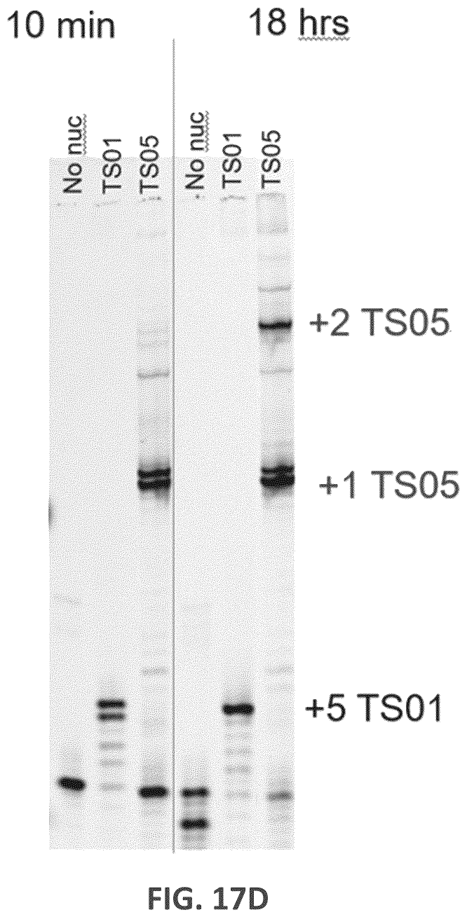

FIGS. 17A-17D are images of electrophoretic gels showing polymerase incorporation of example nucleotides including alpha-imino phosphate groups and bridging sulfurs. More specifically, a polymerase was used to incorporate TS-09 prepared in accordance with FIG. 4E (before deprotecting the amine) and TS-10 prepared in accordance with FIG. 4E (after deprotecting the amine), into a growing polynucleotide based on the sequence of a template polynucleotide. As shown in FIG. 17A, the polymerase tolerated alternative functional groups on the alpha-imino phosphate, and successfully incorporated both the protected amine TS-09 (showing at least one or two incorporations) and the free amine TS-10 (showing at least one, two, or five incorporations). The polymerase was also used to incorporate AS-01 prepared according to FIG. 4C, As shown in FIG. 17B, one, two, and ten of the nucleotide were successfully incorporated into a growing polynucleotide P (upper sequences shown in FIG. 17B) based on the sequence of a template polynucleotide T (lower sequences shown in FIG. 17B). The polymerase was also used to incorporate TS-01 prepared according to FIG. 4A, and GS-01 prepared according to FIG. 4D. As shown in FIG. 17C, five of each of these nucleotides (as well as AS-01) were successfully incorporated into a growing polynucleotide P (upper sequences shown in FIG. 17C) based on the sequence of a template polynucleotide T (lower sequences shown in FIG. 17C).

The polymerase also successfully incorporated CLNs prepared according to operation 910 of FIG. 9. For example, the alkyne groups of TS-01 were coupled to bicyclooctononyne (BCN), and the BCN then reacted with a bulky moiety that included terminal 1,2,4,5-tetrazines (Tz) to form a CLN (designated TS-05). As shown in FIG. 17D, one and two of the CLNs were successfully incorporated.

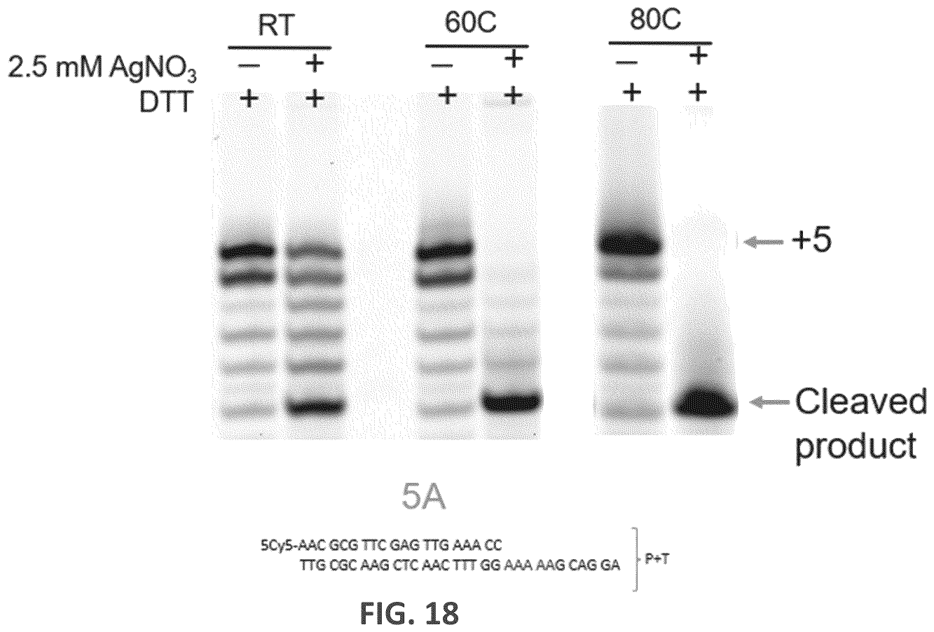

FIG. 18 is an image of an electrophoretic gel showing reagent-caused cleavage of an incorporated example nucleotide including an alpha-imino phosphate group and bridging sulfur. Here, five TS-01 nucleotides prepared in accordance with FIG. 4A were incorporated into a growing polynucleotide P (upper sequence shown in FIG. 18) based on the sequence of a template polynucleotide T (lower sequence shown in FIG. 18). The polynucleotide then was contacted with the cleavage reagent, here 2.5 mM AgNO₃, either at room temperature (RT), 60° C., or 80° C. After one hour of incubation with the cleavage reagent, dithiothreitol (DTT) was added to chelate and deactivate the Ag for gel analysis. From FIG. 18, it may be seen that at each set of reaction conditions, it may be seen that the cleavage reagent generated a cleaved product. Increases in efficiency were observed as temperature increased.

### Additional Comments

It is to be understood that any respective features/examples of each of the aspects of the disclosure as described herein can be implemented together in any appropriate combination, and that any features/examples from any one or more of these aspects can be implemented together with any of the features of the other aspect(s) as described herein in any appropriate combination to achieve the benefits as described herein.

While various illustrative examples are described above, it will be apparent to one skilled in the art that various changes and modifications can be made therein without departing from the invention. The appended claims are intended to cover all such changes and modifications that fall within the true spirit and scope of the invention.

1. A nucleotide, comprising:
a sugar;
a nucleobase coupled to the sugar,
an alpha-imino phosphate group;
a bridging sulfur coupling the alpha-imino phosphate group to the sugar.

2. The nucleotide of claim 1, further comprising a sulfonyl group or an aryl group coupled to the alpha-imino phosphate group.

3. The nucleotide of claim 2, further comprising a first functional group coupled to the alpha-imino phosphate group via the sulfonyl group or aryl group.

4. The nucleotide of claim 2, wherein the aryl group is functionalized.

5. The nucleotide of claim 2, further comprising a second functional group coupled to the nucleobase.

6. (canceled)

7. (canceled)

8. The nucleotide of claim 5, wherein the second functional group further is coupled to the first functional group to form a loop.

9. The nucleotide of claim 5, wherein the nucleobase comprises a purine analog or a pyrimidine analog.

10. The nucleotide of claim 1, wherein the nucleobase comprises a purine, a pyrimidine, a purine analog, or a pyrimidine analog.

11. The nucleotide of claim 1, wherein the sugar comprises ribose or deoxyribose.

12. The nucleotide of claim 1, further comprising at least one phosphate group coupled to the alpha-imino phosphate group.

13. The nucleotide of claim 1, wherein the alpha-imino phosphate group is part of a triphosphate group.

14. A kit, comprising:
the nucleotide of claim 1; and
a reagent for selectively cleaving the bond between the alpha-imino phosphate and the bridging sulfur.

15. The kit of claim 14, wherein the reagent comprises an Ag(I) metal salt, Au(III) metal salt, Pd(II) metal salt, or Oxone.

16. The kit of claim 14, further comprising a polymerase for incorporating the nucleotide into a polynucleotide.

17. A method for modifying a polynucleotide, the method comprising:
using a polymerase to incorporate the nucleotide of claim 1 into a polynucleotide; and
using a reagent to selectively cleave the bond between the alpha-imino phosphate and the bridging sulfur after the nucleotide is incorporated into the polynucleotide.

18. A method of modifying a nucleotide, the method comprising:
installing a thiol group at a 5′ position of a sugar of the nucleotide;
coupling a phosphor atom to the sulfur atom of the thiol group; and
forming an alpha-imino phosphate group coupled to a sulfonyl group or an aryl group.

19. The method of claim 18, wherein installing the thiol group comprises using a Mitsunobu reaction followed by reduction; and wherein forming the alpha-imino phosphate group includes using a phosphoramidite followed by a Staudinger reaction.

20. (canceled)

21. (canceled)

22. The method of claim 18, wherein the sulfonyl group or the aryl group is coupled to a first functional group.

23. The method of claim 18, wherein the nucleotide comprises a nucleobase comprising a second functional group.

24. The method of claim 23, further comprising coupling the first functional group to the second functional group to form a loop.

26. (canceled)
